(19) United States
(12) Patent Application Publication
(10) Pub. No.: US 2025/0161908 A1
(43) Pub. Date: May 22, 2025

(54) Tailored nanoporous sorbents for rapid, noninvasive disease detection

(71) Applicant: National Technology & Engineering Solutions of Sandia, LLC, Albuquerque, NM (US)

(72) Inventors: Nathaniel S. Bobbitt, Albuquerque, NM (US); Michael E. Chandross, Tijeras, NM (US); Dorina F. Sava Gallis, Albuquerque, NM (US); Jason Paul Sammon, Albuquerque, NM (US)

(21) Appl. No.: 18/944,433

(22) Filed: Nov. 12, 2024

Related U.S. Application Data
(60) Provisional application No. 63/599,621, filed on Nov. 16, 2023.

(51) Int. Cl. B01J 20/22; B01D 53/02; B01J 20/28; B01J 20/30; B01J 20/34; G01N 33/497

(52) U.S. Cl. CPC B01J 20/226; B01D 53/02; B01J 20/28066; B01J 20/2808; B01J 20/28083; B01J 20/3085; B01J 20/3425; B01J 20/3483; G01N 33/4975; B01D 2253/204; B01D 2253/304; B01D 2253/306; B01D 2257/708; B01D 2259/40088; B01D 2259/4533

(57) ABSTRACT

Exhaled breath contains trace levels of volatile organic compounds (VOCs) that can reveal information about metabolic processes or pathogens in the body. These molecules can be used for medical diagnosis, but capturing and accurately measuring them is a significant challenge in chemical separations. A highly selective nanoporous metal-organic framework (MOF) sorbent can be used to capture target molecules from a breath sample and pre-concentrate them for use in a detector. As examples, a series of Zr-based MOFs, MOF-818, PCN-777, and UiO-66, each recover around 40-60% of the targets (with the exception of acetaldehyde) at up to 95% relative humidity. In addition, a series of hydrophobic MOFs modified with fluorine functional groups were synthesized that have increased affinity for VOCs in the presence of humidity. In particular, fluorinated MOF-808 derivatives demonstrated substantial increase in the retention of target VOCs adsorbed from a humid gas stream with ppm level VOC concentration.

### CROSS-REFERENCE TO RELATED APPLICATION

This application claims the benefit of U.S. Provisional Application No. 63/599,621, filed Nov. 16, 2023, which is incorporated herein by reference.

### FEDERALLY SPONSORED RESEARCH AND DEVELOPMENT

This invention was made with Government support under Contract No. DE-NA0003525 awarded by the United States Department of Energy/National Nuclear Security Administration. The Government has certain rights in the invention.

### BACKGROUND OF THE INVENTION

Exhaled breath contains trace levels of hundreds of volatile organic compounds (VOCs) that are produced by various metabolic processes in the body, such as cancer, diabetes, or pathogen infection, and make their way into the lungs where they are exhaled. See N. Drabinska et al., J. Breath Res. 15, 034001 (2021). Changes to these metabolic processes, such as infection, organ failure, or other disorders, can alter the profile of VOCs that are exhaled, creating a detectable signal of chemical changes in the body. Therefore, exhaled breath contains a vast wealth of information about the workings of the body. However, breath analysis remains a challenging problem and is not yet routinely used as a medical diagnostic tool. See P. C. Moura et al., Biomed. J. 46, 100623 (2023); I. Oakley-Girvan and S. W. Davis, Cancer Biomarkers 21, 29 (2018); Y. Saalberg and M. Wolff, Clin. Chim. Acta 459, 5 (2016); T. Issitt et al., J. Breath Res. 16, 024001 (2022); and X. Sun et al., Anal. Bioanal. Chem. 408, 2759 (2016).

One challenge is capturing and measuring the VOCs of interest for a particular disease or condition. Deconvoluting the complex signatures of dozens or hundreds of VOCs in exhaled breath to isolate a handful of biomarkers associated with different diseases, infections, or metabolic conditions is a difficult task. The VOCs of interest are generally present in the breath in concentrations at ppb or low ppm levels and are mixed in with many other molecules that are not unique to specific conditions, including competing molecules like water and CO₂. See G. Konvalina and H. Haick, Acc. Chem. Res. 47, 66 (2014); J. D. Fenske and S. E. Paulson, J. Air Waste Manag. Assoc. 49, 594 (1999); and A. Sharma et al., Mol. Diagn. Ther. 27, 321 (2023). It is a significant challenge to isolate and detect only the VOCs of interest for a particular diagnostic question. This is, in essence, a gas phase chemical separation problem of trying to selectively capture and detect particular molecules of interest from a complex mélange of competing molecules. Therefore, there is a need to strategically devise methods to achieve these separations, and furthermore develop design strategies for bespoke separations for future diagnostic needs.

Most previous portable breath analysis technologies can be divided into one of two categories: electronic nose (e-nose) technology and microGC (gas chromatograph). Both of these methods have advantages but also challenges for use as a portable screening tool.

E-nose technology generally relies on differentiated responses to specific chemicals from multiple sensors. See W. Hu et al., Adv. Mater. Technol. 4, 1800488 (2019); and H. Chen et al., IEEE Trans. Biomed. Circuits Syst. 16 (2), 169 (2022). Many groups have used metal-organic frameworks (MOFs) as sensors for electronic noses. See S. Okur et al., Angew. Chem., Int. Ed. 60, 3566 (2021); S. Okur et al., Sens. Actuators B Chem. 306, 127502 (2020); P. Qin et al., J. Mater. Chem. A 10, 25347 (2022); P. Qin et al., Chem. Sci. 12, 15700 (2021); B. A. Day and C. E. Wilmer, ACS Sens. 6, 4425 (2021); J. A. Gustafson and C. E. Wilmer, J. Phys. Chem. C 121, 6033 (2017); and R. Sousa and C. M. Simon, ACS Sens. 5, 4035 (2020). However, the response of these sensors to different gases is rarely perfectly specific to each species. Since the selectivity of MOFs or sensing polymers used for e-noses is not perfect, some competing species are captured by the sorbents. Deconvoluting these mixed and non-orthogonal signals is a difficult problem, and adding more sensors has diminishing returns after about ten sensors. See N. Gantzler et al., J. Phys. Condens. Matter 33, 464003 (2021); Y. S. Kim et al., Sens. Actuators B Chem. 108, 285 (2005); M. C. Lonergan et al., Chem. Mater. 8, 2298 (1996); and E. T. Zellers et al., Anal. Chem. 67, 1092 (1995). This results in both false positives and negatives, which underscores the need for greater selectivity and specificity.

Gas chromatography (GC) is a powerful and well-understood technique widely used in analytical chemistry, often coupled with mass spectrometry (MS). However, GC/MS systems are large and expensive. Portability is essential for the type of widely distributed test that might be used for a pandemic response or in remote areas. Portable chemical analysis systems such as MicroGC or portable ion mobility spectrometry (mini-IMS) are potential solutions. MicroGC employs gas chromatographic separation of analyte molecules through differing affinities for the stationary phase (typically a polymer coating the walls of the column). MicroGC systems produce high specificity with low limits of detection (LOD). The specificity of GC, however, is typically dictated by the length of the separation column which can be a practical issue. For example, Zhou et al. performed in-vivo breath analysis with a portable GC that utilized a 10-meter-long column (coiled up into a small form factor) and experienced significant co-elution (incomplete separation) of target gases. See M. Zhou et al., Anal. Bioanal. Chem. 411, 6435 (2019). A second column was required to achieve complete separation. However, the downside of portable chemical analyzers is that sensitivity is generally lower than lab bench scale instruments. Since breath analysis is targeting very low (ppb-ppm) concentrations of analytes, this could result in inaccurate results. The sensitivity can be improved by using a preconcentrator to capture VOCs from the breath sample prior to the detector.

### SUMMARY OF THE INVENTION

The present invention is directed to a method for breath analysis, comprising providing a preconcentrator comprising one or more metal-organic framework (MOF) sorbents having selectivity for one or more target volatile organic compound (VOC) in a breath, preconcentrating the one or more VOCs on the one or more MOF sorbents, heating the preconcentrator to thermally desorb the preconcentrated one or more target VOCs from the one or more MOF sorbents, and detecting the one or more target VOCs desorbed from the one or more MOF sorbents. For example, the one or more MOF sorbents can comprise a Zr-MOF, such as MOF-808, PCN-777, MOF-818, UiO-66, or NU-1000. For example, the one or more MOF sorbents can comprise a fluorinated MOF.

Grand canonical Monte Carlo (GCMC) simulations were used to select the exemplary zirconium-based MOFs (MOF-808, MOF-818, PCN-777, and UiO-66 with NU-1000) for VOC capture from humid air based on their predicted selectivity with respect to water and CO₂. All five were found to be highly stable after VOC exposure and thermal regeneration. These MOFs were tested for VOC recovery in the presence of humidity, and although humidity significantly reduces the amount of VOC recovered, the MOFs were able to capture significant quantities of each molecule from dilute humid streams. Overall, UiO-66, PCN-777, and MOF-818 had the best performance for recovering VOCs in high humidity, with average recovery above 40% (across the five VOCs tested here) at 95% RH. MOF-808 had exceptional (90%) acetaldehyde recovery up to 60% RH. These results show that highly selective MOFs can capture measurable quantities of VOCs from very humid streams with only ppm levels of the target molecules.

The invention is further directed to a method for synthesizing a fluorinated MOF, comprising providing a MOF with a metal cluster having at least one terminal formate ligand, and reacting the MOF with a fluorine-containing carboxylic acid, whereby at least one of the terminal formate ligands is substituted with a fluorinated function group of the fluorine-containing carboxylic acid. For example, the MOF can comprise MOF-808. For example, the fluorine-containing carboxylic acid can be a fluorinated benzoic acid, 4-fluorobenzoic acid, 3,5-difluorobenzoic acid, 4-(trifluoromethyl)benzoic acid, pentafluorobenzoic acid, or 3,5-bis(trifluoromethyl)benzoic acid. The fluorinated groups are shown to improve the hydrophobicity of the MOF and consequently increased VOC retention from around 60% to about 90%. These hydrophobic variants are useful as sorbents for VOC capture for breath analysis, chemical sensing, and other applications.

### BRIEF DESCRIPTION OF THE DRAWINGS

The detailed description will refer to the following drawings, wherein like elements are referred to by like numbers.

FIG. 1 is a schematic illustration of a method to preconcentrate target VOCs in the exhaled breadth using selective adsorption by MOFs and then detect the thermally desorbed target VOCs.

FIG. 2 is graph comparing heats of adsorption, Q_{ADS}, for water and CO₂ for the 96 Zr-containing MOFs. R2 value for a linear fit=0.86.

FIG. 3A illustrates the MOF-808 extended structure and 6-connected Zr cluster. FIG. 3B illustrates the NU-1000 open channels and 8-connected Zr cluster. FIG. 3C illustrates the UiO-66 porous cage and 12-connected Zr cluster.

FIG. 4 shows N₂ adsorption (closed circles) and desorption (open circles) isotherms of MOFs.

FIG. 5A is a graph showing selectivity for VOCs over water compared to molecular mass of the VOC. FIG. 5B is a graph showing selectivity for VOCs over CO₂.

FIGS. 6A-6C illustrates the topology and linkers of the spn MOFs: MOF-808, PCN-777, and MOF-818, respectively.

FIG. 7A is a graph showing recovery of toluene in five different MOFs at various relative humidity levels. FIG. 7B is a graph showing recovery of octane.

FIG. 7C is a graph showing recovery of acetaldehyde. FIG. 7D is a graph showing recovery of propanal. FIG. 7E is a graph showing recovery of butanone.

FIG. 8A is a graph showing percent recovery of octane for three repeated trials of VOC capture in 40%, 50%, and 60% relative humidity using UiO-66. FIG. 8B is a graph showing percent recovery of acetaldehyde.

FIG. 9 is a graph showing calculated and experimental powder X-ray diffraction (PXRD) of MOFs as synthesized, and post-VOC exposure, indicating that the MOFs are stable.

FIG. 10 shows a schematic illustration the Zr₆ cluster in MOF-808 and the post-synthetic substitution of formates with fluorinated functional groups to provide a functionalized cluster. The fluorinated benzoic acid derivatives (RCOOH) used for the substitution are: F1: 4-fluorobenzoic acid (4-FBA); F2: 3,5-difluorobenzoic acid (3,5-F₂BA); F3: 4-(trifluoromethyl)benzoic acid (4-(CF₃)BA; F5: pentafluorobenzoic acid (F₅BA); and F6: 3,5-bis(trifluoromethyl)benzoic acid (3,5-(CF₃)₂BA).

FIG. 11A shows the chemical structure of the parent MOF-808. FIG. 11B shows the chemical structure of MOF-808 functionalized with F5 (MOF-808-F5) groups.

FIG. 12A shows powder X-ray diffraction (PXRD) of the MOF-808 and MOF-808-F variants. FIG. 12B is a graph of nitrogen isotherms at 77 K adsorption (close symbol) and desorption (open symbol). FIG. 12C shows FT-IR spectra. FIG. 12D shows FT-IR spectra. FIG. 12E is a graph showing thermogravimetric analysis (TGA).

FIG. 13A is a graph showing simulated adsorption isotherms using GCMC at 303.15 K for hexane in MOF-808-F1. FIG. 13B shows simulated adsorption isotherms for acetone in MOF-808-F1. FIG. 13C shows simulated adsorption isotherms for butanone in MOF-808-F5. FIG. 13D shows simulated adsorption isotherms for octane in MOF-808-F5. The percentages indicate the saturation of F-group substitutions on the Zr₆ cluster.

FIG. 14A is a graph showing simulated adsorption isotherms from GCMC for hexane in several MOF-808-F variants, which are all substituted with the same 33% F-group density. FIG. 14B shows simulated adsorption isotherms for octane. FIG. 14C shows simulated adsorption isotherms for acetone. FIG. 14C shows simulated adsorption isotherms for butanone.

FIG. 15A is a graph showing gravimetric adsorption test results for toluene, hexane, acetone, n-propanol, and water in several monofunctional MOF-808-F variants at ambient temperature. FIG. 16B is a graph showing the ratio of VOC to water uptake for each VOC in MOF-808-F variants.

FIG. 16 is a graph showing changes in binding energy for each VOC relative to water for MOF-808, MOF-808-F1, MOF-808-F3, and MOF-808-F5.

FIG. 17A is an illustration of the optimized chemical structure of water in MOF-808. FIG. 17B shows the chemical structure of water in MOF-808-F5. FIG. 17C shows the chemical structure of acetone in MOF-808. FIG. 17D shows the chemical structure of acetone in MOF-808-F5. Relevant nearest adsorbent-MOF distances are indicated.

FIG. 18A is a graph of VOC recovery for MOF-808 and MOF-808-F5 for acetone. FIG. 18B is a graph of VOC recovery for butanone. FIG. 18C is a graph of VOC recovery for propanal. FIG. 18D is a graph of VOC recovery for octane. FIG. 18E is a graph of VOC recovery for acetone. FIG. 18F is a graph of VOC recovery for toluene.

FIG. 19A is a graph of VOC recovery for MOF-808 and MOF-808-F5 for humid and dry butanone. FIG. 19B is a graph of VOC recovery for MOF-808 and MOF-808-F5 for humid and dry octane.

FIG. 20A is a graph of VOC recovery for MOF-808, MOF-808-F5, and two mixed MOF-808-F variants for propanal. FIG. 20B is a graph of VOC recovery for MOF-808, MOF-808-F5, and two mixed MOF-808-F variants for butanone.

FIG. 21 is a graph of VOC recovery for mixture of toluene, octane, and acetaldehyde in MOF-808-F5 compared to recovery for the same VOCs in single-component experiments.

### DETAILED DESCRIPTION OF THE INVENTION

The present invention uses highly selective sorbents made of metal-organic frameworks (MOFs) to capture targeted VOCs. MOFs are nanoporous crystalline structures made from metal (or metal oxide) clusters (or nodes) connected via organic linkers. The chemical space of possible MOFs is enormous and diverse, as these materials can be made from many combinations of clusters and linkers with varying numbers of connections. These components can be arranged in numerous different topologies (connection frameworks) to yield materials with diverse structural properties. As a result, the pore sizes and overall pore structures of MOFs can be systematically varied through the judicious choice of clusters and linkers, tuning the adsorption properties of the materials for specific applications. MOFs have been used for many applications including catalysis, chemical sensing, gas storage, and, notably, separations. See H. Daglar and S. Keskin, Coord. Chem. Rev. 422, 213470 (2020); S. Kitagawa, Chem. Soc. Rev. 43, 5415 (2014); S. K. Firooz et al., Anal. Chim. Acta 149, 340208 (2022); M. S. Denny et al., Nat. Rev. Mater. 1, 1 (2016); A. Bavykina et al., Chem. Rev. 120, 8468 (2020); Ü. Anik et al., Mikrochim. Acta 186, 1 (2019); H. Li et al., EnergyChem 1, 100006 (2019); and H. Li et al., Mater. Today 21, 108 (2018).

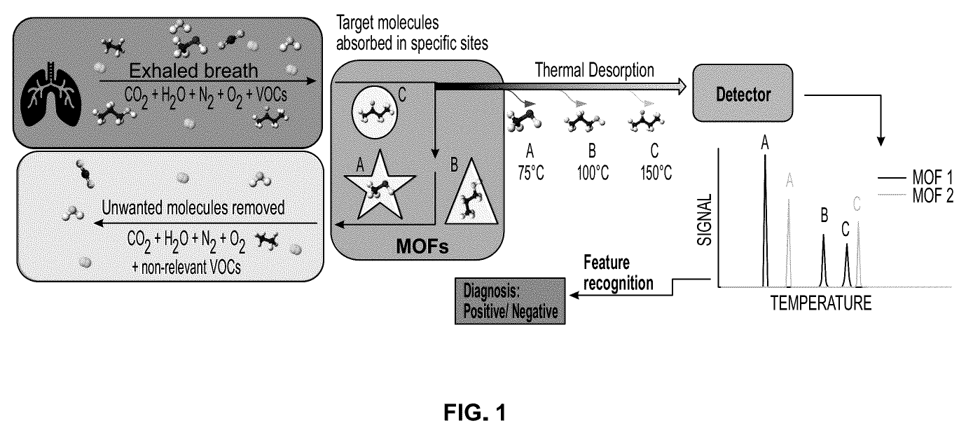

The present invention takes advantage of the high, tunable adsorption selectivity of MOFs by using them as collectors or preconcentrators to capture target VOC molecules out of humid air (e.g., exhaled breath), as shown in FIG. 1. The MOF sorbents (MOF-1 and MOF-2) can then be heated to thermally desorb the target molecules (A, B, and C) that are then sent to a detector. In this scheme, the MOFs serve to selectively separate the target molecules of interest from the breath sample and remove unwanted molecules in order to reduce the number of species confounding detection signals. It also concentrates the VOCs so they can be more easily detected by IMS, GC, or other methods. A selective preconcentrator will achieve some separation via adsorption prior to the target VOCs entering the separation column, reducing the required column length and enabling a smaller and more portable test with a simple detector.

Choosing the correct MOFs to selectively capture the target VOCs of interest is essential. As the design space of possible MOFs is vast, computational screening can be used to narrow down the number of candidate MOFs to a feasible number. There are also practical factors to consider when choosing a MOF for an application. For VOC breath analysis, the MOF will need to be stable in the presence of humidity and able to withstand multiple regeneration cycles of exposure and heating (desorption) so that it can be reused for multiple tests. As an example, the following description focusses on zirconium-based MOFs with zirconium (Zr) clusters because they are known to generally be thermally stable and stable in the presence of water. Some progress has been made in recent years on MOF stability using other metals, but Zr MOFs remain a good choice as resilient MOFs. See K. Ahmad et al., Mater. Sci. Eng. B 262, 114766 (2020); J. H. Cavka et al., J. Am. Chem. Soc. 130, 13850 (2008); C. Gomes Silva et al., Chem. Eur. J. 16, 11133 (2010); J. E. Mondloch et al., Chem. Comm. 50, 8944 (2014); G. Mouchaham et al., Angew. Chem. 127, 13495 (2015); M. Shi et al., Colloids Surf. A Physicochem. Eng. Asp. 602, 125102 (2020); P. Deria et al., J. Am. Chem. Soc. 137, 13183 (2015); O. V. Gutov et al., Chem. Eur. J. 20, 12389 (2014); A. J. Howarth et al., Nat. Rev. Mater. 1, 1 (2016); S. Yuan et al., ACS Cent. Sci. 4, 440 (2018); T. Rasheed, Chemosphere 313, 137607 (2023); and X. Zhang et al., Coord. Chem. Rev. 423, 213507 (2020). Other type of MOFs, such as the Materials of Institute Lavoisier (MIL) and zeolitic imidazolate framework (ZIF) series of MOFs and are good candidates, as they have high chemical and thermal stability, and have been shown to utility for adsorption, gas separation, and chemical sensing.

Breath testing is non-invasive, and this system can provide results rapidly using a compact and portable form factor. Another advantage of this preconcentration method is agility, in that targeting different diseases with different VOC signatures simply requires a set of MOFs tailored to adsorb those specific VOCs. The same detector assembly can then be used in a plug-and-play fashion.

Computational screening can be used to quickly identify MOFs with good selectivity for target VOCs. As an example of the invention, computational modeling was used to select a series of Zr MOFs with promising selectivity for a variety of VOCs. The five MOFs that were indicated by modeling were synthesized and tested in a prototype sensing apparatus, demonstrating the capability to detect trace levels (ppm) of VOCs in realistic humid environments similar to human breath. This demonstrates the feasibility of using tailored MOFs as preconcentrator sorbents for breath analysis.

### Monte Carlo Simulations

Grand canonical Monte Carlo (GCMC) simulations were performed using the multipurpose molecular simulation code RASPA (v2). See D. Dubbeldam et al., Mol. Simul. 42, 81 (2016); and D. Dubbeldam et al., Raspa 2.0: Molecular Software Package for Adsorption and Diffusion in (Flexible) Nanoporous Materials (2021). Surface areas were computed in RASPA using a N₂ probe with Transferable Potentials for Phase Equilibria (TraPPE) parameters for N₂ and Universal Force Field (UFF) parameters for the MOF. Pore sizes were computed in Zeo++ and void fractions were computed in RASPA using a helium probe. See T. F. Willems et al., Microporous Mesoporous Mater. 149, 134 (2012). GCMC simulations for single-component VOC adsorption were performed at 300 K and 0.1 Pa, or approximately 10 ppm VOC concentration at atmospheric conditions. The adsorbates (except for water) were modeled using TraPPE. See B. Chen et al., J. Phys. Chem. B 105, 3093 (2001); M. G. Martin and J. I. Siepmann, J. Phys. Chem. B 102, 2569 (1998); M. G. Martin and J. I. Siepmann, J. Phys. Chem. B 103, 4508 (1999); J. J. Potoff and J. I. Siepmann, AlChE J. 47, 1676 (2001); N. Rai and J. I. Siepmann, J. Phys. Chem. B 111, 10790 (2007); and J. M. Stubbs et al., J. Phys. Chem. B 108, 17596 (2004). Some of the specific molecules are not available in TraPPE but could be constructed from parameters from similar molecules (e.g., butanal is not available in TraPPE but is easily obtained by modifying pentanal). Water was modeled using the TIP3P model. See W. L. Jorgensen et al., J. Chem. Phys. 79, 926 (1983).

For the multicomponent GCMC simulations, selectivity is computed using the binary selectivity formula

$${S}_{i} = ( \frac{{q}_{i}}{{x}_{i}} ) ( \frac{{x}_{j}}{{q}_{j}} ) \qquad ( 1 )$$

where Sᵢ is the selectivity for molecule i over molecule j, qᵢ is the mole fraction of i in the adsorbed phase, xᵢ is the mole fraction of i in the gas phase (reservoir), qⱼ is the mole fraction of j (or total components that are not i) in the adsorbed phase, and xⱼ is the mole fraction of j in the gas phase (or total components that are not i).

### Selection of MOFs

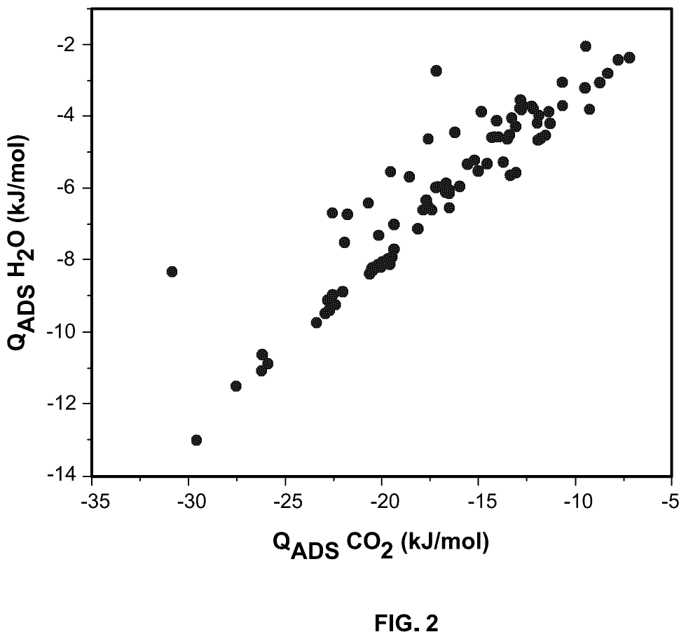

For breath testing applications, the target analyte must be captured from air (breath) that contains high levels of humidity (i.e., water) and CO₂. Therefore, MOFs that exhibit some degree of hydrophobicity are expected to be well-suited to this task. The 96 Zr-containing MOFs from the 2019 CoRE MOF database (all solvent removed, disordered structures removed) were selected for consideration and heats of adsorption and Henry coefficients were computed for water, CO₂, and several diverse VOCs at infinite dilution. FIG. 2 shows the estimated affinities for water and CO₂ from these results for each MOF, with computed heats of adsorption (Q_{ADS}) for water ranging from −2.4 to −13.0 KJ/mol. There is a strong correlation between the heats of adsorption for water and CO₂, implying that more hydrophobic MOFs also have lower affinity for CO₂.

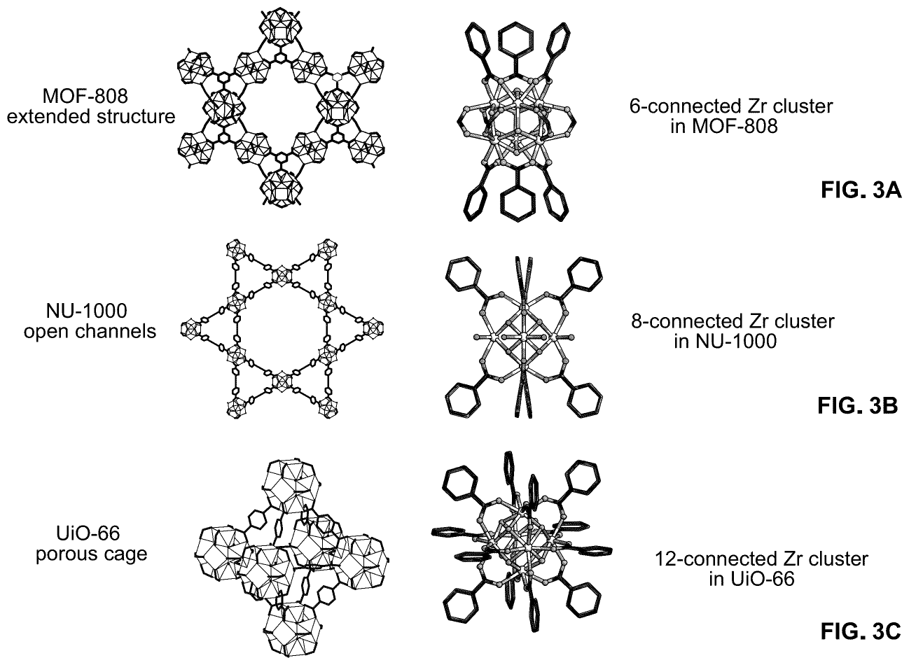

The MOF with lowest affinity for water (−2.4 KJ/mol) is MOF-808. See H. Furukawa et al., J. Am. Chem. Soc. 136, 4369 (2014). MOF-808 is based on hexanuclear oxo/hydroxo Zr⁴⁺ clusters connected through tritopic BTC (1,3,5-benzenetricarboxylate) linkers in the space group Fd-3m. Each organic linker is connected to three inorganic clusters, and each cluster is connected to six linkers in spn topology, as shown in FIG. 3A. Formic acid was used as a modulator for the synthesis of MOF-808, such that the Zr₆ clusters are capped by 6 terminal formate ligands. See J. Baek et al., J. Am. Chem. Soc. 140, 18208 (2018).

The MOFs were also ranked according to the ratio of heat of adsorption of butanone (a target biomarker for COVID-19, among other diseases) to that of water. The highest ranked MOF was PCN-777 (FOTNIN). See D. Feng et al., Angew. Chem. Int. Ed. 54, 149 (2015). PCN-777 has a low heat of adsorption for water (−2.9 KJ/mol), while the heat of adsorption for butanone in PCN-777 is −42.4 KJ/mol, giving a ratio of 15.5 for butanone in water. PCN-777 is isoreticular with MOF-808, sharing the same connectivity and spn topology but with a more extended TATB (4,4′,4″-s-triazine-2,4,6-triyl-tribenzoate) organic linker creating much larger pore diameters than MOF-808 (about 19 Å in MOF-808 vs 33 Å in PCN-777). PCN-777 is also based on 6-connected, hexanuclear Zr⁴⁺ clusters, but these are typically capped by terminal OH⁻/H₂O ligands instead of formate. Only the OH⁻/H₂O-capped version of PCN-777 was analyzed further.

Given that these two MOFs have the same clusters and topology and vary only in the choice of linker, evaluation of both MOF-808 and PCN-777 provides an opportunity to study the effects of pore size and linker functionality (benzene vs. triazine) on adsorption behavior and selectivity. Therefore, another spn Zr₆ MOF, MOF-818, was also studied. See Q. Liu et al., J. Am. Chem. Soc. 141, 488 (2018). This material is less well-studied than MOF-808 and PCN-777 but is an interesting candidate because it is bimetallic, containing trinuclear [Cu₃(μ₃-O)]³⁺ clusters in addition to the same Zr₆ clusters in the other materials. The Cu₃ clusters are capped by 3 ditopic pyrazole-4-carboxylate (PyC) linkers, allowing the Cu₃(PyC)₃ subunits to act as 3-connected supramolecular building units. See C. R. Groom et al., Acta Crystallogr. B. Struct. Sci. Cryst. Eng. Mater. 72, 171 (2016). The simulations indicate the heat of adsorption of water in MOF-818 is −1.9 KJ/mol, close to MOF-808 and PCN-777. The calculated heats of adsorption for CO₂ in the three spn MOFs cover a larger range, with values of −7.4 KJ/mol in MOF-808, −17.8 KJ/mol in PCN-777, and −12.5 KJ/mol in MOF-818.

28 MOFs were rejected due to pore-limiting diameters (PLD) being less than the kinetic diameter of methane (3.8 Å), as this would preclude the adsorption of any VOCs. The MOF with the smallest permissible PLD is UiO-66 (RUBTAK) with a PLD of about 3.9 Å (largest cavity diameter (LCD) of about 8.5 Å), as shown in FIG. 3C. This MOF was included as the limiting case of smallest viable pore size.

NU-1000 was also included in the study, as it is a widely studied Zr⁴⁺ MOF, known to adsorb VOCs, and is highly stable in water. NU-1000 is constructed from 8-connected Zr₆ clusters, as shown in FIG. 3B, and has an intermediate cluster connectivity compared to the spn MOFs (6-connected) and UiO-66 (12-connected). NU-1000 and UiO-66 have computed heats of adsorption for water of −4.5 and −8.7 KJ/mol, respectively, which are both higher than the values for the three spn MOFs. NU-1000 was calculated to have an intermediate heat of adsorption for CO₂ (−14.7 KJ/mol), while the highest heat of adsorption was for UiO-66 (−21.7 KJ/mol). Despite the stronger binding of water and CO₂, NU-1000 and UiO-66 showed very high affinity for the VOCs of interest, suggesting good selectivity over water.

The five selected MOFs, MOF-808, PCN-777, MOF-818, UIO-66, and NU-1000, were synthesized and activated following literature protocols. See G. C. Shearer et al., Chem. Mater. 26, 4068 (2014); J. Baek et al., J. Am. Chem. Soc. 140, 18208 (2018); D. Feng et al., Angew. Chem. Int. Ed. 54, 149 (2015); and Q. Liu et al., J. Am. Chem. Soc. 141, 488 (2019). The corresponding organic linkers are widely available commercially.

### MOF Properties and Characterization

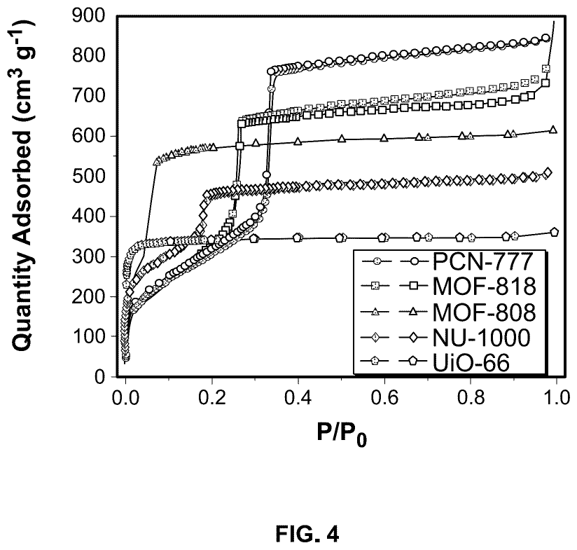

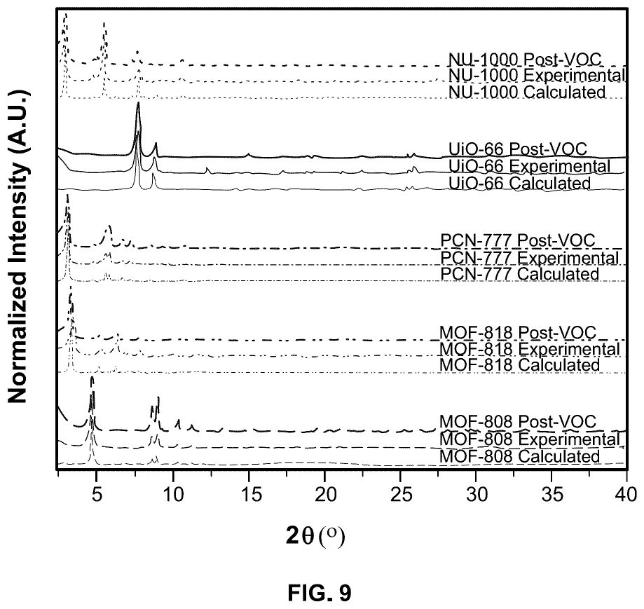

Textural properties for the selected MOFs were computed using Zeo++ with a radius of 1.86 Å which corresponds to a N₂ probe. Results are presented in Table 1. The MOFs were thoroughly characterized using PXRD and nitrogen adsorption, as shown in FIG. 9 (labeled “Experimental” and “Calculated”) and FIG. 4.

Table 1. Textural properties of selected MOFs. LCD is the largest cavity diameter, PLD is the pore limiting diameter, LFS is the largest free sphere, VF is the void fraction, and SA is the surface area.

| | LCD | PLD | LFS | | SA | SA | density |
|---|---|---|---|---|---|---|---|
| | (Å) | (Å) | (Å) | VF | (m²/g) | (m²/cm³) | (g/cm³) |
| MOF-808 | 19.1 | 14.2 | 19.1 | 0.71 | 1616 | 1335 | 0.826 |
| PCN-777 | 33.6 | 27.7 | 33.5 | 0.89 | 3050 | 832 | 0.273 |
| MOF-818 | 32.4 | 26.1 | 32.4 | 0.86 | 2141 | 848 | 0.142 |
| UiO-66 | 8.4 | 4.0 | 8.4 | 0.46 | 1110 | 1420 | 1.280 |
| NU-1000 | 29.7 | 29.0 | 29.6 | 0.81 | 3254 | 1537 | 0.472 |

### Single-Component Simulation Data

GCMC simulations were used to predict the adsorption of a diverse set of VOCs, including alkanes, alkenes, alcohols, ketones, and aromatics, on the five selected MOFs. Single component calculations were performed in RASPA at 300 K and 10 ppm, to approximate the concentration of target VOCs in exhaled breath. See J. D. Fenske and S. E. Paulson, J. Air Waste Manag. Assoc. 49, 594 (1999). The results of this screening are shown in Table 2. GCMC simulations were also performed at 100 ppm (not shown).

The GCMC simulations predict that NU-1000 and UiO-66 should adsorb the highest quantities of VOCs at 10 ppm. This is attributed to strong van der Waals interactions between the VOC adsorbates and the confined pore environment of UiO-66 and the smaller micropores of NU-1000, leading to favorable binding energies and high uptake values. The spn MOFs (MOF-808, PCN-777, and MOF-818) generally showed lower calculated VOC adsorption but still showed good selectivity for the target VOCs over water. The five Zr-MOFs were predicted to adsorb similar quantities of water, with MOF-818 showing somewhat higher affinity than the other materials. On the other hand, the NU-1000 and UiO-66 models gave significantly higher CO₂ uptake than the spn MOFs. Water and CO₂ are present in high concentrations in breadth relative to VOCs and can saturate potential VOC binding sites, implying that engineering high VOC selectivity into preconcentrator materials can be just as important as maximizing their absolute VOC binding affinities. Ultimately, high selectivity for the target VOCs with respect to both water and CO₂ is necessary for realistic applications. It is noted that the pores in UiO-66 are quite small and could result in transport limitations, despite the excellent selectivity.

TABLE 2
Results of GCMC simulations for VOC adsorption in MOFs at 300K
and 10 ppm concentration. Units in mmol adsorbate per kg MOF.

| | MOF- | | | | |
|---|---|---|---|---|---|
| VOC (10 ppm) | 808 | MOF-818 | PCN-777 | NU-1000 | UiO-66 |
| methane | 0.001 | 0.002 | 0.003 | 0.008 | 0.020 |
| ethane | 0.003 | 0.003 | 0.005 | 0.093 | 0.852 |
| propane | 0.008 | 0.005 | 0.011 | 0.705 | 31.953 |
| butane | 0.019 | 0.008 | 0.023 | 6.794 | 417.432 |
| pentane | 0.048 | 0.014 | 0.048 | 85.643 | 911.764 |
| octane | 0.494 | 0.083 | 0.557 | 2372.030 | 625.681 |
| propene | 0.007 | 0.004 | 0.010 | 0.452 | 12.805 |
| 1-butene | 0.015 | 0.006 | 0.019 | 3.739 | 273.984 |
| 2-cis-butene | 0.018 | 0.008 | 0.023 | 6.335 | 142.036 |
| 2-trans-butene | 0.017 | 0.007 | 0.022 | 4.322 | 477.849 |
| cyclohexane | 0.084 | 0.018 | 0.084 | 227.840 | 1583.427 |
| benzene | 0.139 | 0.031 | 0.088 | 142.466 | 1307.667 |
| toluene | 0.385 | 0.064 | 0.210 | 1339.514 | 2129.899 |
| acetone | 0.069 | 0.038 | 0.030 | 4.022 | 388.219 |
| butanone | 0.191 | 0.077 | 0.064 | 47.573 | 1113.725 |
| ethanal | 0.025 | 0.021 | 0.010 | 0.233 | 3.642 |
| propanal | 0.056 | 0.037 | 0.021 | 1.751 | 145.476 |
| butanal | 0.154 | 0.073 | 0.043 | 18.309 | 987.428 |
| methanol | 0.019 | 0.019 | 0.005 | 0.039 | 0.192 |
| ethanol | 0.049 | 0.035 | 0.012 | 0.314 | 6.878 |
| 1-propanol | 0.124 | 0.060 | 0.024 | 2.549 | 221.653 |
| 2-propanol | 0.086 | 0.050 | 0.020 | 1.451 | 154.212 |
| CO₂ | 0.002 | 0.003 | 0.004 | 0.020 | 0.078 |
| water | 0.005 | 0.012 | 0.003 | 0.002 | 0.002 |

### Multicomponent Simulation Data

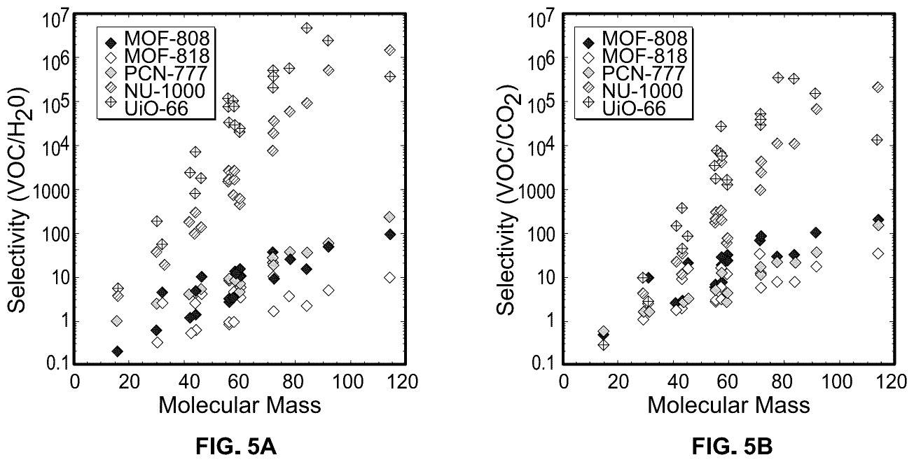

Any application using breath testing will include significant quantities of water vapor and CO₂ in the sample. It is therefore critical that the sorbent material used to capture the target VOCs have high selectivity in the presence to these competing molecules. To help understand the impact of competitive adsorption on VOC uptake, GCMC simulations were performed with the selected VOCs, water, and CO₂ in ratios that approximate the composition of exhaled breath (10 ppm VOC and 50000 ppm or 5% each of water and CO₂). The results of these calculations are shown in FIGS. 5A and 5B. It is noted that in many cases more water and CO₂ are adsorbed than VOCs in absolute terms, but due to the highly dilute level of VOCs in the simulation, the selectivity for VOCs is high.

The selectivities of each MOF for each VOC with respect to water and CO₂ are given in Table 3. NU-1000 and UiO-66 have the highest selectivity from simulation, with many values being 10²-10⁶, while selectivities of MOF-808, PCN-777, and MOF-818 generally range from 1 to about 100. All five MOFs are generally more selective for the VOCs over water and CO₂, suggesting that these materials can be used effectively as sorbents to capture VOCs from breath. The main exceptions are methane and ethane. This is unsurprising given the small size of these molecules and the weakness of their dispersion interactions, implying that light hydrocarbons may be less useful as biomarker molecules compared to other (larger) VOCs.

The most notable changes between the single component and multicomponent adsorption are for 1-propanol, which is reduced by more than 50% in all five MOFs in the presence of water and CO₂, and for methanol, which is increased in all five MOFs when water and CO₂ are present (e.g., by 6% in UiO-66 and by 29% in NU-1000). The latter is likely due to increased adsorbate-adsorbate interactions between methanol and water.

TABLE 3
VOC selectivity with respect to water and CO₂. Results
from multicomponent GCMC simulations are 300K with 10
ppm VOC and 50000 ppm CO₂ and water.

| | | MOF- | MOF- | PCN- | NU- | UiO- |
|---|---|---|---|---|---|---|
| | Selectivity | 808 | 818 | 777 | 1000 | 66 |
| 1-butene | VOC/total | 3.8 | 1.3 | 6.4 | 332.6 | 7014.4 |
| | VOC/H₂O | 2.7 | 0.8 | 8.3 | 1551.7 | 74175.6 |
| | VOC/CO₂ | 6.0 | 2.9 | 5.2 | 186.3 | 3681.2 |
| 1-propanol | VOC/total | 14.7 | 5.1 | 3.5 | 105.9 | 2324.4 |
| | VOC/H₂O | 10.6 | 3.3 | 4.5 | 445.9 | 19679.2 |
| | VOC/CO₂ | 23.6 | 11.9 | 2.9 | 60.1 | 1235.2 |
| 2-cis- | VOC/total | 4.5 | 1.4 | 7.4 | 565.5 | 3340.4 |
| butene | | | | | | |
| | VOC/H₂O | 3.3 | 0.9 | 9.5 | 2587.7 | 32906.5 |
| | VOC/CO₂ | 7.2 | 3.2 | 6.0 | 317.5 | 1759.5 |
| 2-propanol | VOC/total | 21.1 | 9.5 | 5.4 | 137.5 | 3053.5 |
| | VOC/H₂O | 15.4 | 6.0 | 7.0 | 616.5 | 24728.8 |
| | VOC/CO₂ | 33.7 | 22.2 | 4.4 | 77.3 | 1627.2 |
| 2-trans- | VOC/total | 4.4 | 1.3 | 7.0 | 384.4 | 14974.2 |
| butene | | | | | | |
| | VOC/H₂O | 3.2 | 0.9 | 9.0 | 1685.2 | 117922.3 |
| | VOC/CO₂ | 7.0 | 3.1 | 5.7 | 216.9 | 7994.7 |
| acetone | VOC/total | 18.0 | 7.1 | 9.3 | 370.1 | 10969.3 |
| | VOC/H₂O | 13.1 | 4.5 | 12.1 | 1637.1 | 76708.5 |
| | VOC/CO₂ | 28.7 | 16.4 | 7.6 | 208.6 | 5907.0 |
| benzene | VOC/total | 34.9 | 5.8 | 28.3 | 12647.0 | 86107.4 |
| | VOC/H₂O | 25.2 | 3.7 | 36.6 | 58325.7 | 587447.7 |
| | VOC/CO₂ | 56.6 | 13.5 | 23.1 | 7092.4 | 46458.6 |
| butanal | VOC/total | 40.7 | 14.8 | 14.1 | 1700.7 | 60010.1 |
| | VOC/H₂O | 29.5 | 9.4 | 18.3 | 7569.8 | 212191.8 |
| | VOC/CO₂ | 65.2 | 34.7 | 11.5 | 957.9 | 34946.7 |
| butane | VOC/total | 4.8 | 1.4 | 7.7 | 606.4 | 12224.8 |
| | VOC/H₂O | 3.5 | 0.9 | 9.9 | 2702.4 | 98965.1 |
| | VOC/CO₂ | 7.7 | 3.2 | 6.3 | 341.5 | 6514.8 |
| butanone | VOC/total | 51.1 | 15.3 | 20.9 | 4351.1 | 89705.2 |
| | VOC/H₂O | 37.3 | 9.7 | 27.0 | 19336.3 | 369275.3 |
| | VOC/CO₂ | 81.3 | 35.9 | 17.1 | 2451.3 | 51053.6 |
| ethanal | VOC/total | 6.3 | 3.9 | 3.3 | 21.3 | 83.0 |
| | VOC/H₂O | 4.6 | 2.5 | 4.3 | 98.8 | 801.0 |
| | VOC/CO₂ | 10.3 | 9.1 | 2.7 | 12.0 | 43.8 |
| ethane | VOC/total | 0.8 | 0.5 | 1.9 | 7.9 | 18.4 |
| | VOC/H₂O | 0.6 | 0.3 | 2.5 | 36.5 | 182.8 |
| | VOC/CO₂ | 1.3 | 1.1 | 1.6 | 4.4 | 9.7 |
| ethanol | VOC/total | 14.0 | 6.7 | 4.1 | 29.4 | 160.3 |
| | VOC/H₂O | 10.1 | 4.2 | 5.3 | 133.5 | 1751.7 |
| | VOC/CO₂ | 22.7 | 15.9 | 3.3 | 16.5 | 84.0 |
| methane | VOC/total | 0.3 | 0.3 | 0.8 | 0.8 | 0.5 |
| | VOC/H₂O | 0.2 | 0.2 | 1.0 | 3.8 | 5.3 |
| | VOC/CO₂ | 0.5 | 0.7 | 0.6 | 0.5 | 0.3 |
| methanol | VOC/total | 5.7 | 3.9 | 2.0 | 4.4 | 5.5 |
| | VOC/H₂O | 4.2 | 2.5 | 2.6 | 18.5 | 53.8 |
| | VOC/CO₂ | 9.1 | 9.2 | 1.6 | 2.5 | 2.9 |
| octane | VOC/total | 130.4 | 15.3 | 185.4 | 368260.9 | 24246.0 |
| | VOC/H₂O | 94.9 | 9.7 | 240.0 | 1594705.2 | 374845.6 |
| | VOC/CO₂ | 208.6 | 36.0 | 151.0 | 208166.1 | 12528.2 |
| pentane | VOC/total | 12.2 | 2.5 | 15.7 | 7581.4 | 52099.3 |
| | VOC/H₂O | 8.9 | 1.6 | 20.3 | 35805.1 | 514854.9 |
| | VOC/CO₂ | 19.3 | 6.0 | 12.8 | 4239.5 | 27437.9 |
| propanal | VOC/total | 15.7 | 7.2 | 7.0 | 163.7 | 3415.6 |
| | VOC/H₂O | 11.4 | 4.6 | 9.0 | 745.0 | 28036.9 |
| | VOC/CO₂ | 25.2 | 16.9 | 5.7 | 91.9 | 1818.6 |
| propane | VOC/total | 1.9 | 0.9 | 3.6 | 62.8 | 685.5 |
| | VOC/H₂O | 1.4 | 0.6 | 4.6 | 293.7 | 7059.8 |
| | VOC/CO₂ | 3.0 | 2.0 | 2.9 | 35.1 | 360.3 |
| propene | VOC/total | 1.6 | 0.8 | 3.1 | 40.4 | 271.8 |
| | VOC/H₂O | 1.2 | 0.5 | 4.1 | 186.1 | 2512.9 |
| | VOC/CO₂ | 2.6 | 1.8 | 2.6 | 22.6 | 143.7 |
| toluene | VOC/total | 65.3 | 7.7 | 44.7 | 119238.7 | 297364.5 |
| | VOC/H₂O | 47.4 | 4.9 | 57.6 | 516801.2 | 2536033.2 |
| | VOC/CO₂ | 104.6 | 18.0 | 36.4 | 67394.1 | 157942.0 |
| cyclo- | VOC/total | 20.7 | 3.4 | 27.3 | 20007.0 | 600513.6 |
| hexane | | | | | | |
| | VOC/H₂O | 15.0 | 2.1 | 35.3 | 90882.5 | 4786563.8 |
| | VOC/CO₂ | 33.6 | 8.0 | 22.2 | 11240.8 | 320352.2 |

### Comparison of spn Topology MOFs

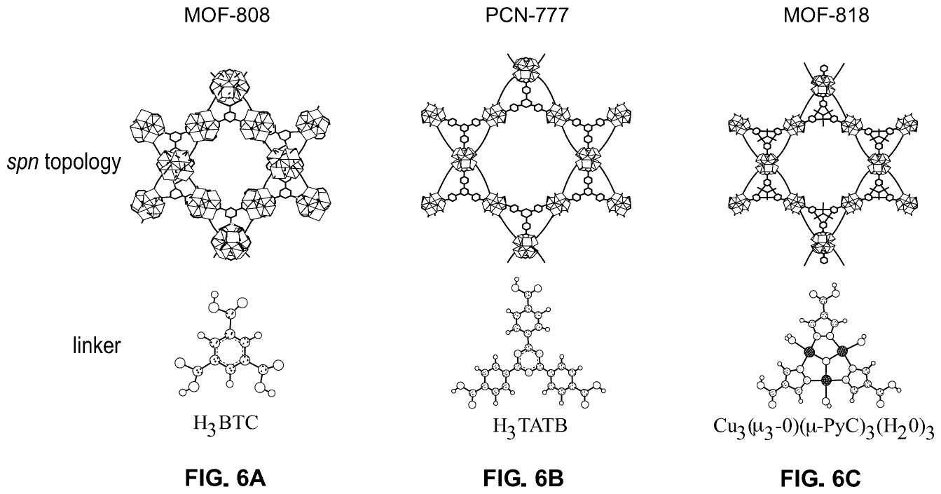

As MOF-808, PCN-777, and MOF-818 have the same Zr₆ clusters and network topology, as shown in FIGS. 6A-6C, the calculated selectivity was compared between them. From the GCMC simulations, PCN-777 is generally the most selective for nonpolar alkanes and alkenes, while MOF-808 is more selective for the polar VOCs like alcohols and ketones. MOF-808 is also more selective for the aromatics benzene and toluene. The selectivity of MOF-808 is attributed to its smaller pore size that results in a strong electrostatic potential. Note that while benzene and toluene are generally considered non-polar, the TraPPE model used contain charges. The EQeq charge calculations also show slightly more charge separation between the clusters and linkers in MOF-808 compared to PCN-777. In MOF-808 the Zr cluster (including all Zr and O atoms) has a net charge of −5.92e, compared to −5.89e in PCN-777. The average charge on a single Zr⁴⁺ ion in MOF-808 is 2.13e, compared to 1.95e in PCN-777. The average charge per Zr is calculated as 2.16e in MOF-818, but this gives an incomplete picture of the charge environment in the pore due to the presence of Cu²⁺ as a heterometal on the linkers.

The stronger electrostatic interactions in MOF-808 (compared to PCN-777) is clear from decomposing the MOF-adsorbate interaction energy into van der Waals (vdW) and Coulombic components, as shown in Table 4. Adsorption of nonpolar hydrocarbons (i.e., alkanes, alkenes) is entirely driven by vdW interactions in all five MOFs, as the Coulombic contributions are minimal in each case. For the other VOCs, the breakdown of vdW vs. Coulombic contributions to adsorption energy varied widely. Coulombic interactions are generally more significant in MOF-808 and MOF-818, while vdW forces dominate in PCN-777. This can be attributed to the lower degree of charge separation between the cluster and linker in PCN-777 that creates a less polar pore environment than MOF-808 and MOF-818. For example, the percentage of Coulombic adsorption energy of acetone in MOF-808 is 31.1%, while in PCN-777 it is only 8.2%. Similarly, the adsorption energy of 1-propanol in MOF-808 is 51.5% Coulombic compared to 18.2% in PCN-777, and the adsorption of butanal in MOF-808 is 34.3% Coulombic compared to 9.9% in PCN-777.

The largest Coulombic contributions to VOC adsorption are seen for MOF-818, which has the highest charge per Zr. Additionally, the incorporation of both Cu²⁺ and Zr⁴⁺ clusters further increases the ionic character of its pore environment. Accordingly, GCMC simulations indicate that MOF-818 shows the least affinity for nonpolar VOCs and the highest affinity for water, while falling between MOF-808 and PCN-777 for the polar VOCs.

TABLE 4
Energy decomposition for adsorbates in MOFs. These values are the percent
of average total energy for the MOF-adsorbate interactions attributed
to Coulomb interactions (Coul) and van der Waal interactions or dispersion
(vdW). Adsorbate-adsorbate interactions are not included here. Some values
are negative or greater than 100 due to repulsive interactions.

| | MOF-808 | | PCN-777 | | MOF-818 | | NU-1000 | | UiO-66 | |
|---|---|---|---|---|---|---|---|---|---|---|
| | Coul | vdW | Coul | vdW | Coul | vdW | Coul | vdW | Coul | vdW |
| benzene | 7.5 | 92.5 | 0.8 | 99.2 | 12.7 | 87.3 | 0.2 | 99.8 | −0.6 | 100.6 |
| toluene | 4.4 | 95.6 | 0.0 | 100.0 | 9.8 | 90.2 | 0.2 | 99.8 | −0.9 | 100.9 |
| acetone | 31.2 | 68.8 | 8.3 | 91.7 | 46.7 | 53.3 | 1.4 | 98.6 | 1.3 | 98.7 |
| butanone | 26.6 | 73.4 | 6.6 | 93.4 | 42.4 | 57.6 | 0.9 | 99.1 | 0.8 | 99.2 |
| ethanal | 43.8 | 56.2 | 15.6 | 84.4 | 60.4 | 39.6 | 3.2 | 96.8 | 2.7 | 97.3 |
| propanal | 39.0 | 61.0 | 12.1 | 87.9 | 53.7 | 46.3 | 2.4 | 97.6 | 1.8 | 98.2 |
| butanal | 34.3 | 65.7 | 9.9 | 90.1 | 49.5 | 50.5 | 2.0 | 98.0 | 1.9 | 98.1 |
| methanol | 62.1 | 37.9 | 29.6 | 70.4 | 74.4 | 25.6 | 5.3 | 94.7 | 4.5 | 95.5 |
| ethanol | 56.5 | 43.5 | 23.6 | 76.4 | 67.5 | 32.5 | 3.7 | 96.3 | 2.2 | 97.8 |
| 1-propanol | 51.5 | 48.5 | 18.2 | 81.8 | 61.2 | 38.8 | 2.9 | 97.1 | 2.1 | 97.9 |
| 2-propanol | 52.1 | 47.9 | 17.0 | 83.0 | 62.6 | 37.4 | 2.8 | 97.2 | 1.3 | 98.7 |
| CO₂ | 23.7 | 76.3 | 7.0 | 93.0 | 23.1 | 76.9 | 2.4 | 97.6 | 3.3 | 96.7 |
| water | 85.4 | 14.6 | 61.9 | 38.1 | 120.8 | −20.8 | 23.2 | 76.8 | 12.1 | 87.9 |

Notably, the percentage of Coulomb contribution for both NU-1000 and UiO-66 is low, generally less than 5% for most VOCs. These MOFs have 8 and 12 linkers, respectively, connected to each cluster, reducing access to binding sites for polar VOCs to interact with the Zr⁴⁺. This results in these interactions being dominated by electrostatics. Both benzene and toluene have a small negative Coulomb contribution in UiO-66, indicating the electrostatic contribution is repulsive.

### Humid Vapor Testing

The synthesized MOFs were exposed to N₂ streams containing variable humidity percentages and 20 ppm of the specified VOCs for 60 minutes. The MOFs were then heated to desorb VOC adsorbates, which were then detected and quantified using GC-FID. Importantly, no VOCs were detected by the flame ionization detector (FID) until the MOF samples were heated, indicating that the MOFs retain adsorbed VOCs completely at ambient temperature until the heat is applied.

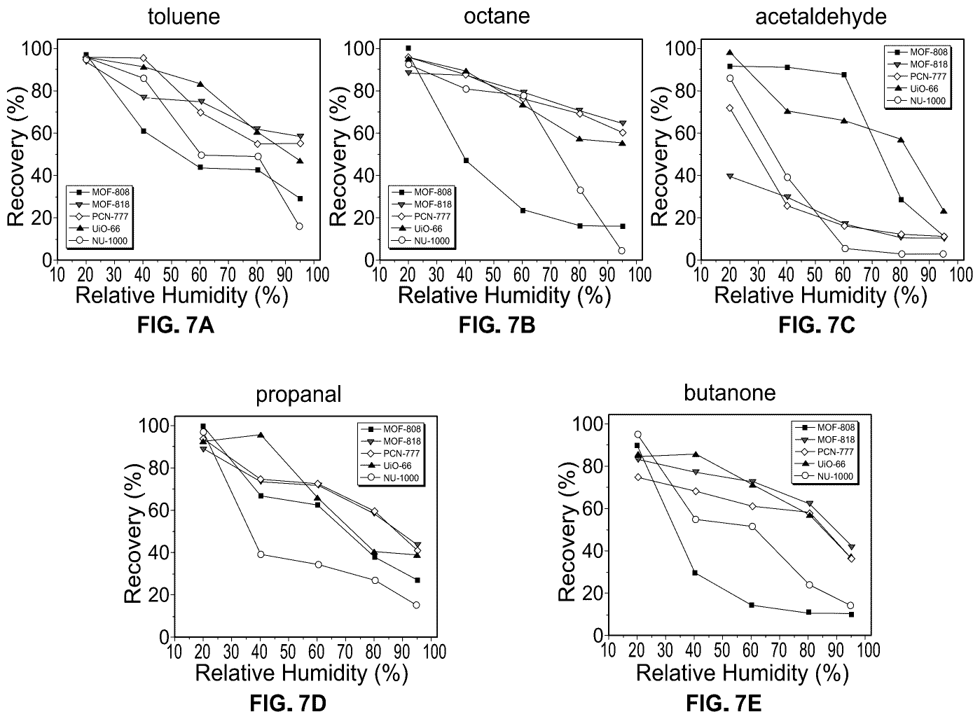

FIGS. 7A-7E show the results of the VOC/humidity capture testing for toluene, octane, acetaldehyde, propanal, and butanone in the MOFs at 20, 40, 60, 80, and 95% relative humidity (RH). The percent recovery is computed as the fraction of the VOC mass desorbed from the MOF during the heating step (as determined by GC-FID) compared to the total mass that was passed over the MOF. These recovery percentages were assumed to generally reflect the amounts of VOCs captured by the MOF, with higher percentages indicating higher VOC uptake.

The VOC recovery percentages were found to generally decrease as relative humidity was increased, as expected due to competition for adsorption sites between water and the VOCs. However, the effects of humidity are complex and vary between different VOCs and MOFs. One striking example is the difference in acetaldehyde uptake between MOF-808 and NU-1000 (FIG. 7C). Notably, at 60% humidity MOF-808 recovered nearly 90% of the acetaldehyde it was exposed to, while NU-1000 only recovered 5%. Both materials showed decreased acetaldehyde adsorption at higher humidities, but MOF-808 consistently outperformed NU-1000 for this VOC. Conversely, MOF-808 showed very low adsorption of octane at humidities greater than 20%, with the four other materials showed significantly higher octane recovery percentages in most cases (FIG. 7B). This directly corroborates the computational results, as MOF-808 was predicted to have higher affinity for more polar VOCs.

UiO-66 was consistently one of the best performing materials at high humidities, again agreeing with the simulation results. UiO-66 recovered 40-60% of all VOCs at 80% humidity, including 57% for acetaldehyde. MOF-818 and PCN-777 also had good performance at high humidities with the exception of acetaldehyde. These MOFs recovered above 50% of the other four VOCS at 80% humidity. NU-1000 has generally poor performance compared to the other MOFs for any VOC at 80 or 95% RH.

These results indicate that acetaldehyde is especially difficult to capture from humid streams, with several MOFs recovering less than 20% above 40% humidity. This is a particularly difficult separation, as acetaldehyde is a small polar molecule similar to water. The best performing MOFs above 80% RH are UiO-66 followed by MOF-808. Notably MOF-808 has very high recovery for acetaldehyde up to 60% RH, but then drops significantly at higher humidity.

For butanone, the best MOFs are MOF-818, PCN-777, and UiO-66, which perform similarly. This agrees with the screening based on Henry coefficients that predicted PCN-777 to have the highest butanone affinity.

NU-1000 tends to perform poorly compared to the other four MOFs. This is significant because NU-1000 was chosen as a benchmark MOF for comparison, not based on data from the computational screening. However, NU-1000 remains an interesting case as the GCMC simulations suggest it should have high selectivity for most VOCs over water. The experiments indicate that the other MOFs have better recovery for all five VOCs tested but this discrepancy is likely due to the hierarchical pore structure in NU-1000. The smaller micropore yields very high VOC selectivity, but becomes saturated with prolonged exposure, and the selectivity of the mesopore is not as high, lowering the overall performance.

Generally, the experimental trends agree with the simulations. The results suggest that UiO-66 should have overall strong performance, and this is attributed to the small pores that induce strong van der Waal interactions. MOF-808 was predicted to have stronger Coulomb interactions than PCN-777, and consequently seems to generally be more negatively affected by humidity.

Based on these results, it appears that VOC recovery suffers significantly above 60% RH, even in the best performing MOFs. This leads to a challenge for any kind of VOC-based breath testing. However, even at 95% RH the MOFs capture detectable levels of the VOCs, indicating that using MOFs to capture VOC biomarkers from humid breath is viable. The level of humidity in exhaled breath varies significantly, ranging from 42-91%, and the MOFs presented here are generally effective in that regime. See E. Mansour et al., Sens. Actuators B Chem. 304, 127371 (2020).

### MOF Stability and Reuse

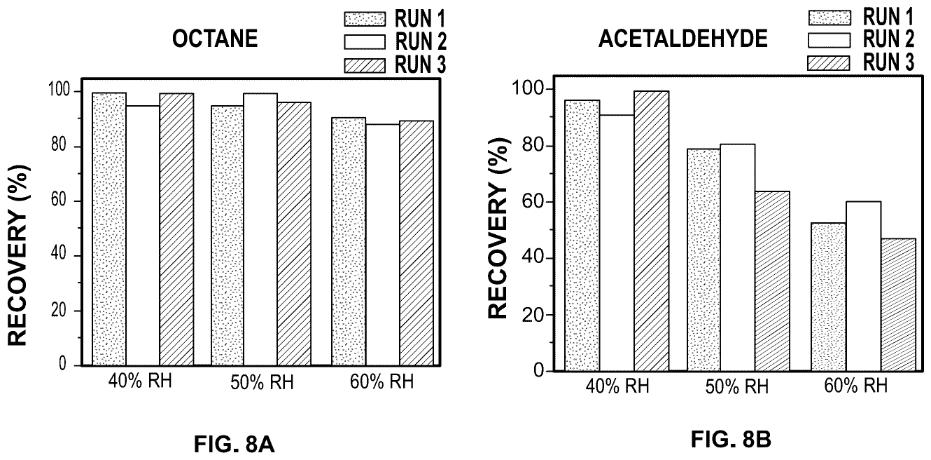

In order to be practically useful as VOC collectors, the MOFs are preferably able to be regenerated and reused with no loss of selectivity. To test recyclability, octane and acetaldehyde in UiO-66 was chosen as a representative system because UiO-66 has strong performance for several of the VOCs. A UiO-66 sample was subjected to three consecutive adsorption/desorption cycles at 40%, 50%, and 60% relative humidity (for a total of 9 cycles per VOC). The recovery percentages for the three individual runs showed little change at each humidity level for both VOCs, as shown in FIGS. 8A and 8B. These experiments indicate that UiO-66 is reusable as a preconcentrator material and gives reproducible results. The MOF samples used for the VOC capture studies were recovered after the exposures and were found by PXRD to remain highly crystalline in each case, as shown in FIG. 9 (labeled “Post-VOC”), despite the wide range of VOCs and humidities each material encountered. This validates the stability of the Zr⁴⁺ MOFs for VOC capture applications.

### Fluorinated MOFs with Improved Hydrophobicity

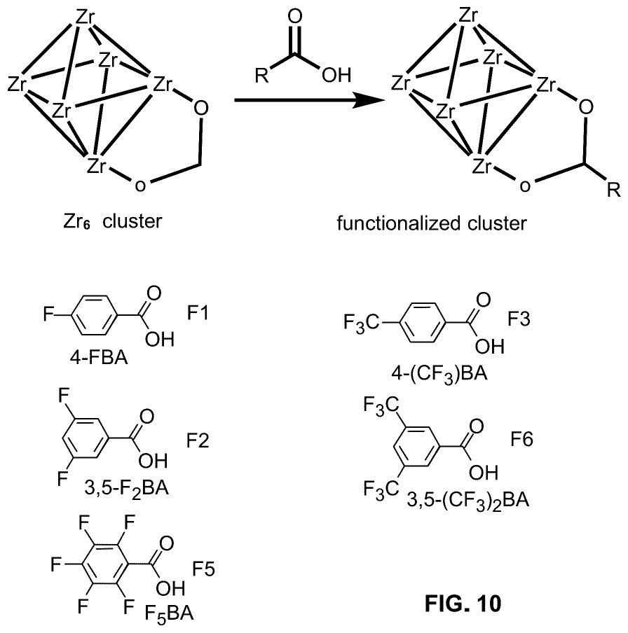

One of the great benefits of MOFs is their ability to be customized via choices of different clusters, linkers, and also, post-synthetic addition of functional groups. Post-synthetic modification is a convenient way to improve adsorption or chemical properties of MOFs that are already known to be stable and have existing synthesis protocols. See S. Mandal et al., Adv. Funct. Mater. 31, 2006291 (2021); Z. Wang and S. M. Cohen, Chem. Soc. Rev. 38, 1315 (2009); and M. Kalaj and S. M. Cohen, ACS Cent. Sci. 6, 1046 (2020). As described above, humidity can severely hinder VOC adsorption in MOF materials. It has been known for some time that fluorine groups added to MOFs or other sorbents will increase the hydrophobicity, with perfluorinated alkanes (PFAS) being a notable example. See K. Jayaramulu et al., Adv. Mater. 31, 1900820 (2019); and S. Mukherjee et al., Trends Chem. 3, 911 (2021). Therefore, it was hypothesized that adding fluorine groups would make the MOFs more hydrophobic and improve the selectivity for target VOCs. MOF-808 was used as a platform because it is a highly stable, robust MOF with a scalable synthesis procedure, and amenable to post-synthetic modification. See and C. Ardila-Suárez et al., CrystEngComm 21, 3014 (2019); and X. Meng et al., CrystEngComm 21, 3146 (2019). As can be seen in FIG. 3A, the Zr₆ cluster in MOF-808 is occupied by only 6 linkers, leaving the remaining 6 equatorial sites occupied by terminal formate ligands available for functionalization. FIG. 10 shows a schematic illustration the Zr₆ cluster in MOF-808 with a formate ligand and the post-synthetic substitution of the formate with a fluorinated functional group to provide a functionalized cluster. As examples, MOF-808-Fs were synthesized with five different fluorinated functional groups. Each group is labeled F1, F2, etc. corresponding to the number of F atoms in the group. However, the formate ligands can be substituted with a wide variety of fluorine-containing aliphatic and aromatic carboxylic acids coordinating directly to the metal cluster, such as perfluoroheptanoic acid; pentafluoropropionic acid; 2,3,6-Trifluorophenylacetic acid; 2,3,3,3-Tetrafluoropropanoic acid; 3,4-Difluorophenylacetic acid; (3,4,5-Trifluorophenyl) acetic acid; and 2,4,6-trifluorophenylacetic acid.

### Synthesis of Fluorinated MOFs

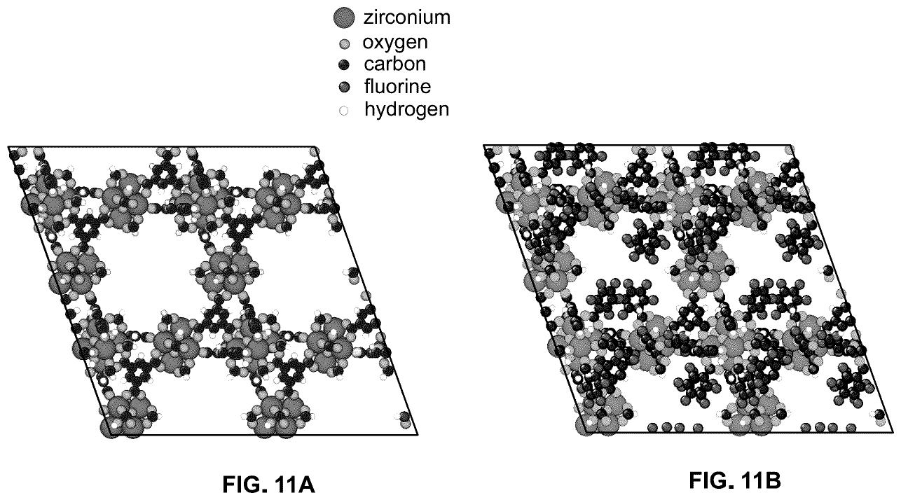

To synthesize MOF-808, 1,3,5-benzenetricarboxylic acid (BTC; 0.210 g, 1.00 mmol) and zirconyl chloride octahydrate (ZrOCl₂·8H₂O; 0.970 g, 3.01 mmol) were added to 250 mL screw-top jar with a PTFE-lined lid. The solids were dissolved through sonication in a mixture of 30 mL N,N-dimethylformamide (DMF) and 30 mL formic acid. The reaction mixture was heated in an isothermal oven to 100° C. at 1.5° C./min, held at 100° C. for 24 hours, then cooled to room temperature at 1° C./min. A white, powdery solid formed and was collected by centrifugation and washed with DMF (3×25 mL) and acetone (3×25 mL) in a 50 mL centrifuge tube. The solid was then activated in a vacuum oven at 85° C. for 12 hours prior to use. FIG. 11A shows the chemical structure of the parent MOF-808.

To synthesize the fluorinated MOF-808-Fs, MOF-808 (0.150 g, 0.66 mol) and a fluorinated benzoic acid FX (FX=F1—0.4623 g, F2—0.5217 g, F3—0.6274 g, F5—0.6998 g, F6—0.8518, 3.3 mmol) were added to a 125 mL screw-top jar with a PTFE-lined lid, followed by 25 mL of DMF. The jar was sealed, and the reaction was sonicated for 30 seconds to mix but not dissolve the solid. The reaction was heated to 60° C. in an isothermal oven for 18 hours. The solid was collected by centrifugation and washed with DMF (3×25 mL) and acetone (3×25 mL), then dried at 85° C. under vacuum for 12 hours. FIG. 11B shows the chemical structure of MOF-808-F5 functionalized with F5 groups.

### Characterization of Fluorinated MOFs

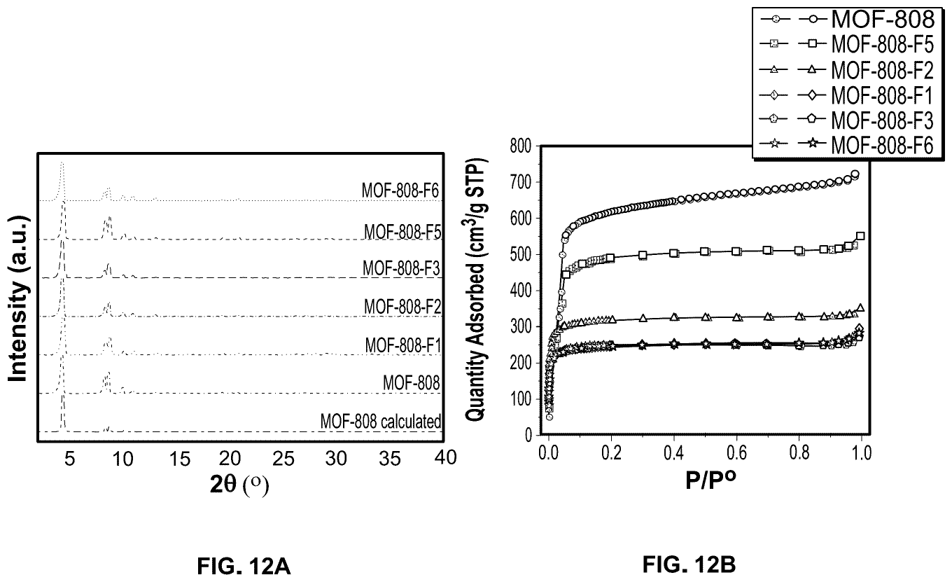

FIG. 12A shows powder X-ray diffraction (PXRD) patterns of the MOF-808 and MOF-808-F variants. FIG. 12B is a graph of nitrogen adsorption isotherms at 77 K adsorption (close symbol) and desorption (open symbol). FIG. 12C shows FTIR spectra of MOF-808 and MOF-808-F variants. FIG. 12D is a zoomed version of FIG. 12C at 1800−500 wavenmbers. FIG. 12E is a graph showing thermogravimetric analysis (TGA). Activated samples were heated to 900° C. at 10° C./min. in air.

### Monte Carlo Simulations of Fluorinated MOFs

The Julia software package PoreMatMod was used to in silico add functional groups to the MOF-808 structure. See E. A. Henle et al., J. Chem. Inf. Model. 62, 423 (2022). The MOF-808 unit cell contains 4 Zr₆ clusters (or secondary building units, SBUs) each with 6 formates for a total of 24 potential substitution sites. Fluorinated MOFs were created with 4, 8, and 12 substitutions, equaling 17%, 33%, and 50% coverage of the formate sites. The resulting library of structures then underwent geometry optimization using density functional theory (DFT) in VASP and subsequently assigned partial charges via the DDEC6 method. See G. Kresse and J. Furthmüller, Comput. Mater. Sci. 6, 15 (1996); G. Kresse and J. Furthmüller, Phys. Rev. B 54, 11169 (1996); G. Kresse et al., Phys. Rev. B 50, 13181 (1994); and J. P. Perdew et al., Phys. Rev. Lett. 77, 3865 (1996). Grand canonical Monte Carlo (GCMC) simulations were performed in RASPA v2.0. See D. Dubbeldam et al., Raspa 2.0: Molecular Software Package for Adsorption and Diffusion in (Flexible) Nanoporous Materials (2021).

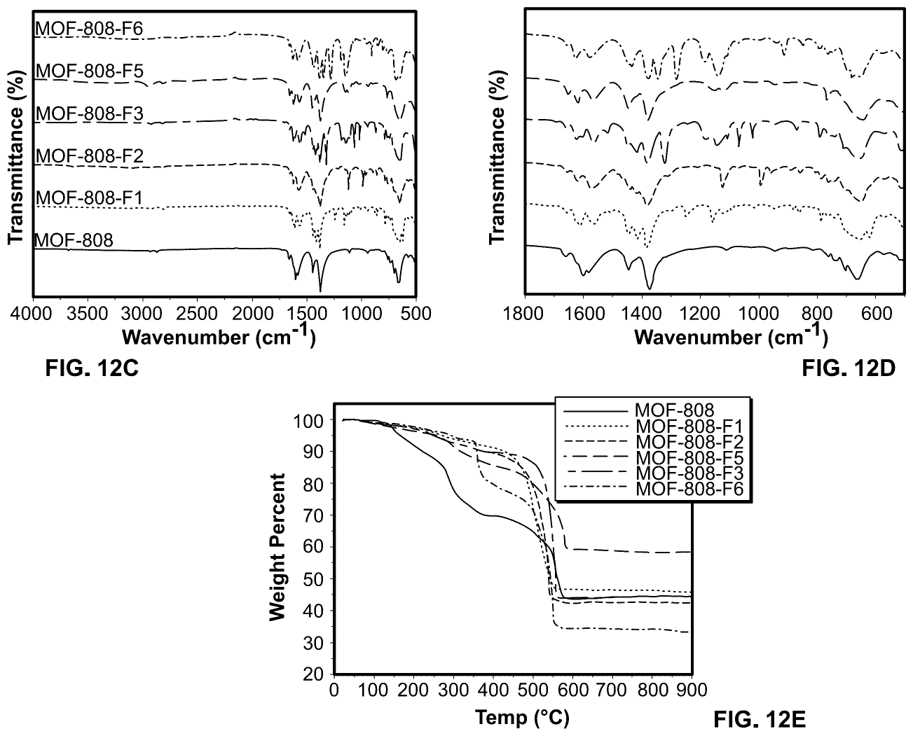

GCMC simulations were performed on MOF-808 functionalized with the five fluorinated groups for adsorption of acetone, hexane, octane, butanone, and octane. Results for hexane, acetone, butanone, and octane are shown in FIGS. 13A-13D for MOF-808-F1 and MOF-808-F5 with varying density of F-group substitutions. A consistent trend was found that as the density of substitutions increases, the VOC uptake at saturation decreases. This is a clear consequence of a reduction in free volume due to adding more functional groups. However, for applications such as VOC capture or sensing where the expected VOC concentration is very low, the uptake at low pressure is more important than the saturation level. A consistent trend was also seen that adding more functional groups shifts the step of the isotherm to lower pressures, indicating stronger adsorption interaction. This trend is seen across different types of F groups and VOCs. This is corroborated by the trend in computed enthalpy of adsorption for MOF-808 and MOF-808-F5 shown in Table 5.

TABLE 5
Computed enthalpy of adsorption for selected
VOCs in MOF-808 and MOF-808-F5. Percentages
represent F-group density. Values in kJ/mol.

| | MOF- | MOF-808-F5 | | |
|---|---|---|---|---|
| VOC | 808 | 17% | 33% | 50% |
| 1-propanol | −17.6 | −23.9 | −25.4 | −28.8 |
| acetaldehyde | −18.3 | −21.4 | −23.1 | −51.4 |
| acetone | −21.0 | −27.2 | −28.7 | −35.4 |
| butanone | −22.8 | −30.9 | −32.4 | −35.3 |
| hexane | −19.3 | −28.6 | −30.8 | −33.2 |
| octane | −21.2 | −30.3 | −33.1 | −39.2 |
| propanal | −17.1 | −22.6 | −24.2 | −29.3 |
| water | −10.1 | −11.5 | −12.6 | −14.8 |
| toluene | −35.0 | −47.4 | −47.1 | −47.4 |

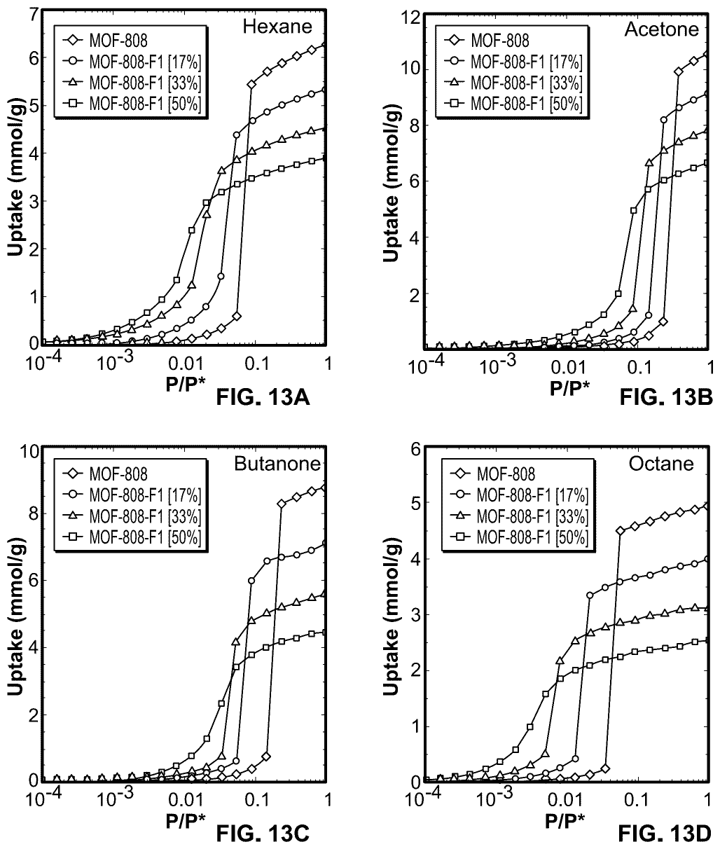

FIGS. 14A-14D show more simulated isotherms for hexane, acetone, butanone, and octane, respectively, in parent MOF-808 and all five F-group variants. These MOFs are all substituted at 33% density meaning they have an average of 2 F-groups per Zr₆ cluster randomly distributed through the unit cell. Again, a consistent trend is seen that adding F-groups shifts the isotherm step towards lower pressure. There is also a reduction saturation uptake that correlates to the size of the group, e.g. MOF-808-F6, which has two bulky methyl groups, is consistently the lowest saturation. However, this trend does not appear to apply to the shift in the step between different types of groups. Rather, the various shifted steps appear to be at about the same pressure for each respective VOC, with no clear trend between the various F-groups. For this reason, it was hypothesized that MOF-808-F5 could be the most interesting material to pursue due to the balance between maximizing F density and pore free volume. This is corroborated by the trend in computed enthalpy of adsorption for MOF-808 and the MOF-808-F variants shown in Table 6.

TABLE 6
Computed enthalpy of adsorption for selected VOCs
in MOF-808 derivatives. Values in kJ/mol.

| | MOF- | MOF-808- | MOF-808- | MOF-808- | MOF-808- |
|---|---|---|---|---|---|
| VOC | 808 | F1 | F2 | F5 | F6 |
| 1-propanol | −17.6 | −27.6 | −26.7 | −25.4 | −26.7 |
| acetaldehyde | −18.3 | −23.1 | −25.4 | −23.1 | −25.4 |
| acetone | −21.0 | −31.0 | −32.8 | −28.7 | −32.8 |
| butanone | −22.8 | −36.5 | −35.1 | −32.4 | −35.1 |
| hexane | −19.3 | −38.5 | −31.8 | −30.8 | −31.8 |
| octane | −21.2 | −40.1 | −32.5 | −33.1 | −32.5 |
| propanal | −17.1 | −25.9 | −27.1 | −24.2 | −27.1 |
| water | −10.1 | −12.0 | −13.2 | −12.6 | −13.2 |
| toluene | −35.0 | −58.1 | −48.1 | −47.1 | −48.1 |

The computed heats of adsorption corroborated the trends found in the isotherms. Addition of the functional groups increases the enthalpy of adsorption, which agrees with the notable shift of the isotherm step to lower pressures. Also, as the density of the F groups increases (more substitutions), the enthalpy of adsorption generally becomes stronger. The enthalpy of adsorption for water likely also increases, but less than the other VOCs which suggests the selectivity should shift to favor VOC adsorption.

### Gravimetric Adsorption Tests

A simple test was preformed to assess the gravimetric adsorption of VOCs in the MOF-808-F variants at room temperature by exposing the MOFs to VOC vapor at ambient conditions and measuring the change in mass. All five variants with monofunctional substitutions as well as several mixed bifunctional and trifunctional MOFs were tested, to see if there are synergistic effects from combining F-groups.

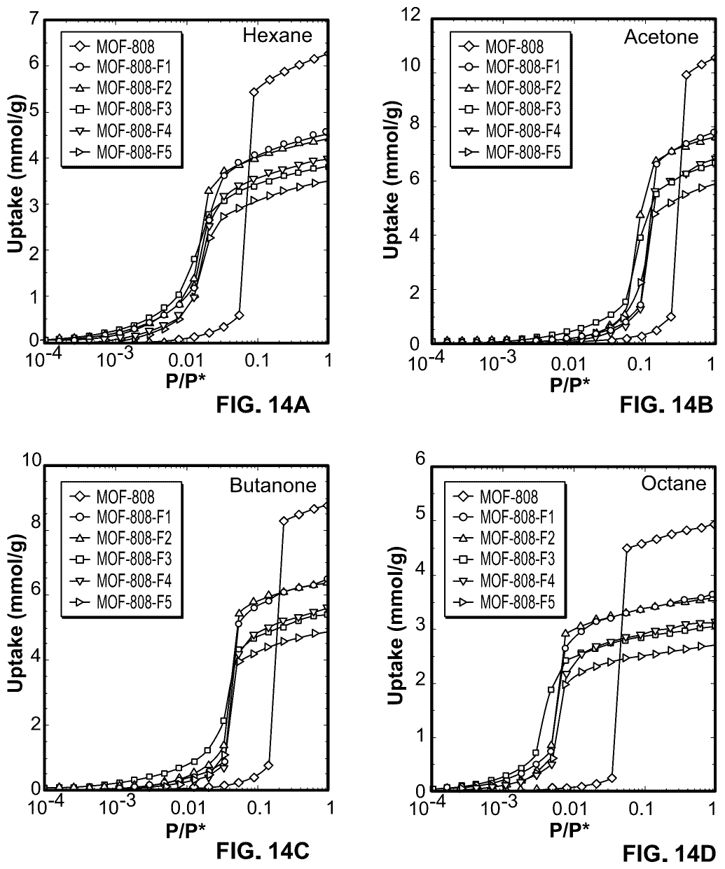

As shown in FIG. 15A, generally, the amount of VOC uptake in the MOF-808-F variants is lower than the parent MOF-808, with the notable exception of acetone in MOF-808-F5, which shows a 3-fold increase from 1 mg/mg to 3 mg/mg acetone adsorption. Notably, the amount of water adsorbed in all five MOF-808-F variants decreases, indicating increased hydrophobicity. FIG. 15B shows the ratio of VOC uptake (in mg) to water uptake (in mg) for each VOC and MOF. This indicates that in some cases, even though the total VOC adsorption is lower in the MOF-808-F variants, the selectivity for the VOC is increased. This is the case for acetone in all the MOF-808-F variants, especially MOF-808-F5. MOF-808-F3, MOF-808-F5, and MOF-808-F6 also show increased selectivity for hexane. There are also slight increases for toluene and n-propanol selectivity in some of the MOF-808-F variants.

Several bi-functional and tri-functional MOFs with various combinations of the F-groups were also synthesized and tested. No clear synergy was observed between different mixed functional groups. Notably, the bi-functional groups containing the F5 group (MOF-808-F1F5 and MOF-808-F2F5) did show significant increases in acetone adsorption, similar to the monofunctional MOF-808-F5. However, the monofunctional MOF-808-F5 shows a larger increase, so it was concluded there is little benefit to mixing F-groups and the monofunctional MOF-808-F5 is probably the best candidates. Based on this simple test, the exceptional acetone adsorption in MOF-808-F5 suggests that this MOF will be a good candidate for further tests in the VOC recovery experiments.

### Density Functional Theory

To develop a molecular level understanding of VOC-MOF interactions, DFT simulations were performed of VOCs and water binding in the MOF pore and studied the influence of the F-groups. Specifically, the binding energy of water, acetone, hexane, propanal, 1-propanol, and toluene was calculated in MOF-808, MOF-808-F1, MOF-808-F3, and MOF-808-F5. The structures used for these calculations each had 33% substitution (average 2 functional groups per cluster). The binding energies were determined by

$$Binding Energy = {E}_{M O F} + {E}_{a d s orbent} - {E}_{M O F + a d s orbent} \qquad ( 2 )$$

where E_{MOF}, E_{adsorbent}, and E_{MOF+adsorbent} are the energies of the optimized MOF structure, gas-phase adsorbent, and adsorbent in the MOF structure, respectively. In this notation, positive energies indicate energetically favorable binding geometries. Three specific binding sites were explored, namely the μ₃-OH, fully-coordinated metal site, and the F-groups. Using the raw calculated binding energy for each MOF-adsorbent pair the maximum (most favorable) binding energy across all binding sites was determined. Binding energies for acetone, hexane, propanal, 1-propanol, toluene, and water were calculated in MOF-808, MOF-808-F1, MOF-808-F3, and MOF-808-F5.

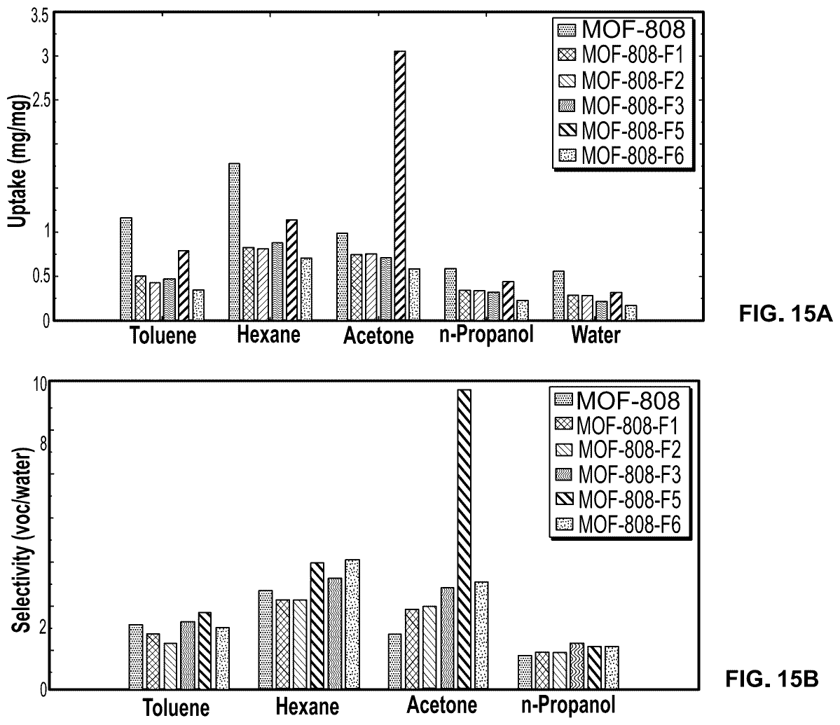

To showcase the increase or decrease for each VOC from water, a change in binding energy was then calculated, given by

$${Δ}_{B E} = B {E}_{VOC} - B {E}_{water} \qquad ( 3 )$$

where BEᵢ^{VOC} and BEᵢ^{Water} are the calculated binding energies for a given VOC and water. The calculated changes in binding energy are shown in FIG. 16. It is seen that the binding energy for each VOC is higher than that of water, excluding toluene in MOF-808. Moreover, the Δ_{BE} increases for almost all VOCs in fluorinated MOF-808 structures, peaking in MOF-808-F3 for acetone, 1-propanol, and toluene, indicating the selectivity shifts more to VOCs with respect to water.

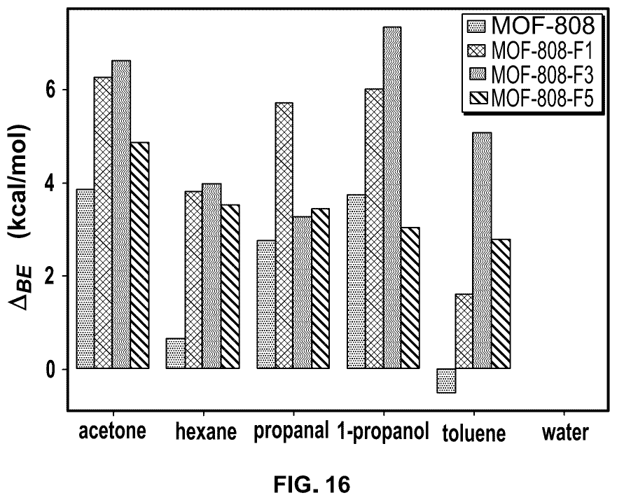

To explore this effect further, the optimized geometries of acetone and water in MOF-808 and MOF-808-F5 at the μ₃-OH site were examined. Note that this is the most energetically favorable binding site for these two molecules in these two MOFs. Structural images are shown in FIGS. 17A-17D. When acetone and water are adsorbed to MOF-808 (FIGS. 17A and 17C) there is a single interaction between the O-atom in the molecule and the MOF cluster via the μ₃-OH. However, when bound to MOF-808-F5 there are several more interactions that become relevant between the F atoms on the substitutions and the various H atoms in the molecules (FIGS. 17B and 17D). These interactions lead to generally higher binding energies in the fluorinated MOFs. More interactions between the F atoms on the substitutions and the acetone molecule are observed than are seen for water due to the increase in the size of the molecule and the availability of H atoms. Thus, even though water has a shorter and likely stronger bond to the MOF cluster itself, the increased interactions between the F atoms and acetone lead to higher overall binding energies for the VOCs when compared to water. This indicates that the F atoms in the substitutions are playing a large role in the selective binding of VOCs when compared with water.

### VOC Recovery Experiments

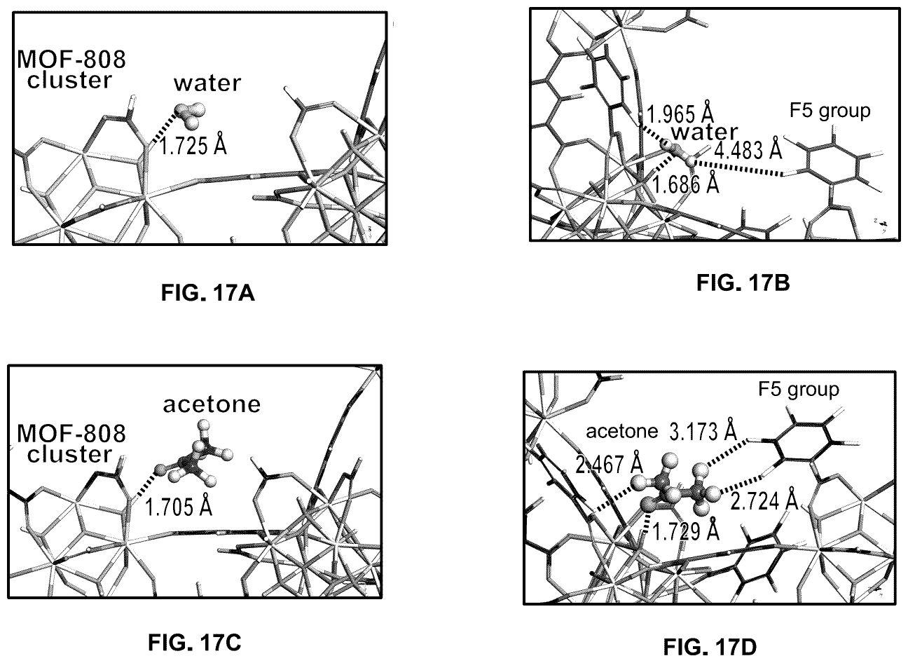

The performance of MOF-808-F5 compared to parent MOF-808 was tested by exposing the MOFs to 20 ppm vapor in humid N₂ and measuring the mass of VOC recovered. The results in FIG. 18A-18F show significant improvement in MOF-808-F5 for all the VOCs tested, except for acetaldehyde. Acetaldehyde is notably a small polar molecule much like water, and the increased hydrophobicity likely also has related effect of reducing acetaldehyde adsorption. Designing a sorbent to separate acetaldehyde from water is a challenge, and therefore, acetaldehyde may not be the most useful biomarker due to the difficulty of capturing it from humid air. However, substantial improvement was found for recovery of butanone, propanal, octane, acetone, and toluene. Notably, MOF-808-F5 recovers 92% of propanal at 95% relative humidity, as shown in FIG. 18C.

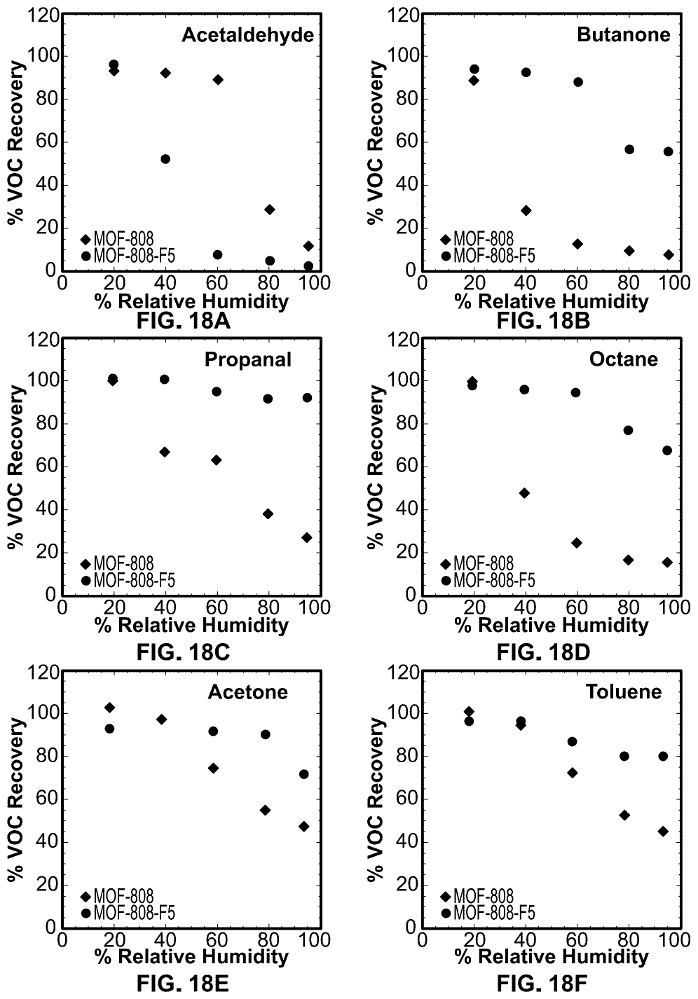

A commercial desiccant (a mixture of Dryrite and Molsieve 13×) was also used in the line as a barrier prior to the humid VOC gas reaching the MOF. This is a practical measure that can be used to remove water in the sample stream. Including the desiccant does improve the VOC recovery at high humidity, as shown in FIGS. 19A and 19B, although recovery does still decrease at high humidity suggesting the desiccant did not capture 100% of the water from the stream. Therefore, there is still sensitivity to humidity. Notably, the VOC recovery of MOF-808 with the desiccant is similar to MOF-808-F5 without the desiccant. Depending on the requirements of a given application, this could mean that the parent MOF-808 used in conjunction with a desiccant is a good choice for VOC recovery. Alternately, if a desiccant cannot be used in a given context (perhaps due to space restraints, etc.), then MOF-808-F5 provides equally good VOC recovery without using a desiccant.

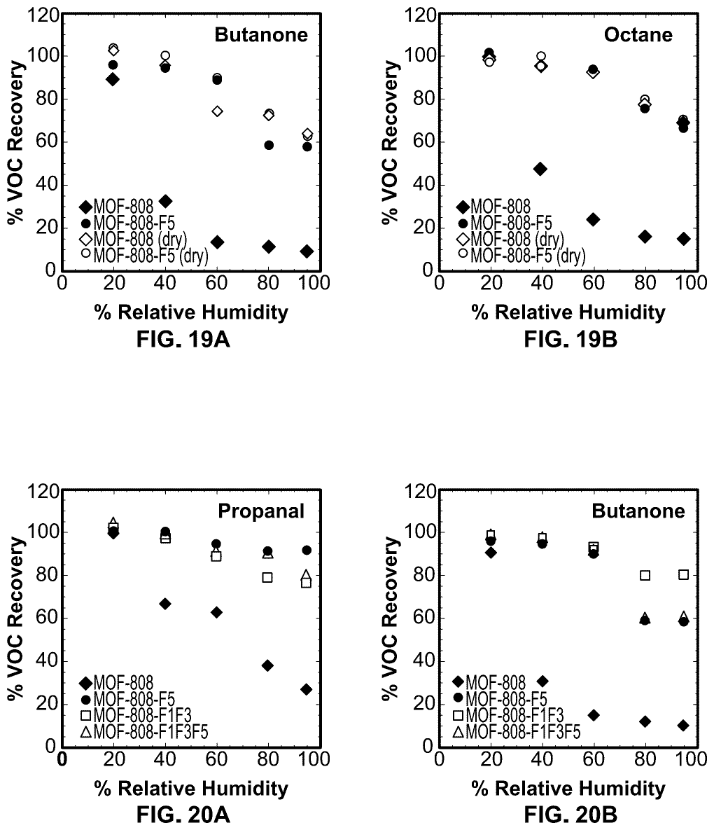

The VOC recovery with two mixed MOF-808-F variants: MOF-808-F1F3 and MOF-808-F1F3F5 was also tested, as shown in FIGS. 20A and 20B. While there is clear improvement over the recovery of MOF-808, again there is no clear advantage to the mixed functional groups. In some cases, such as butanone at 95% RH, MOF-808-F1F3 outperforms MOF-808-F5 (80% vs 57% butanone recovery) but for propanal, MOF-808-F5 outperforms both multifunctional variants (92% recovery vs 77-79% recovery at 95% RH). In most cases, the two multifunctional variants are about the same as MOF-808-F5, but all three are notably better than parent MOF-808 (except for acetaldehyde).

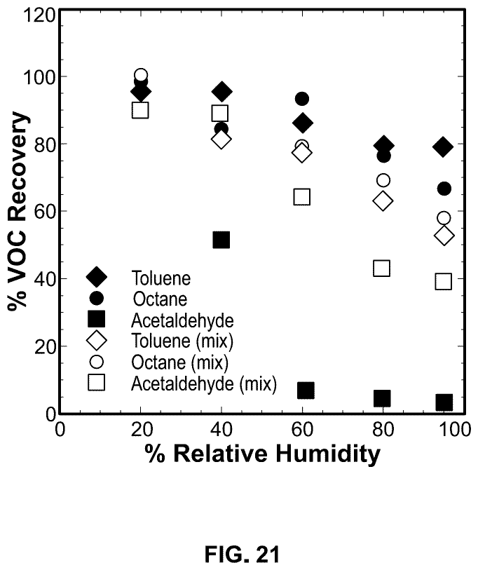

Finally, MOF-808-F5 was tested with a mixture of VOCs in a multicomponent experiment. The vapor stream contained 20 ppm each of toluene, octane, and acetaldehyde, along with varying levels of humidity. Results are shown in FIG. 21.

The present invention has been described as tailored nanoporous metal-organic framework sorbents for rapid, noninvasive disease detection. It will be understood that the above description is merely illustrative of the applications of the principles of the present invention, the scope of which is to be determined by the claims viewed in light of the specification. Other variants and modifications of the invention will be apparent to those of skill in the art.

1. A method for breath analysis, comprising:
providing a preconcentrator comprising one or more metal-organic framework (MOF) sorbents having selectivity for one or more target volatile organic compound (VOC) in a breath,
preconcentrating the one or more VOCs on the one or more MOF sorbents,
heating the preconcentrator to thermally desorb the preconcentrated one or more target VOCs from the one or more MOF sorbents, and
detecting the one or more target VOCs desorbed from the one or more MOF sorbents.

2. The method of claim 1, wherein the one or more MOF sorbents comprises a Zr-MOF.

3. The method of claim 2, wherein the Zr-MOF comprises MOF-808, PCN-777, MOF-818, UIO-66, or NU-1000.

4. The method of claim 1, wherein the one or more MOF sorbents comprises a fluorinated MOF.

5. A method for synthesizing a fluorinated metal-organic framework (MOF), comprising
providing a MOF with a metal cluster having at least one terminal formate ligand, and
reacting the MOF with a fluorine-containing carboxylic acid, whereby at least one of the terminal formate ligands is substituted with a fluorinated function group of the fluorine-containing carboxylic acid.

6. The method of claim 5, wherein the MOF comprises a Zr-MOF.

7. The method of claim 6, wherein the Zr-MOF comprises MOF-808.

8. The method of claim 5, wherein the fluorine-containing carboxylic acid comprises a fluorinated benzoic acid.

9. The method of claim 8, wherein the fluorinated benzoic acid comprises 4-fluorobenzoic acid, 3,5-difluorobenzoic acid, 4-(trifluoromethyl)benzoic acid, pentafluorobenzoic acid, or 3,5-bis(trifluoromethyl)benzoic acid.
